(19) United States
(12) Patent Application Publication
(10) Pub. No.: US 2026/0053521 A1
(43) Pub. Date: Feb. 26, 2026

(54) NOVEL TONGUE-CLEANER HEAD ADAPTED FOR ATTACHMENT TO COUPLING HANDLE OF PREVAILING ELECTRIC TOOTHBRUSH FOR TURNING SAME TO ADVANTAGEOUS ELECTRIC TONGUE-CLEANER

(71) Applicant: Patrick Alphonso, Ann Arbor, MI (US)

(72) Inventor: Patrick Alphonso, Ann Arbor, MI (US)

(21) Appl. No.: 19/369,157

(22) Filed: Oct. 24, 2025

Related U.S. Application Data
(60) Provisional application No. 63/686,845, filed on Aug. 25, 2024.

(51) Int. Cl. A61B 17/24; A61B 17/00

(52) U.S. Cl. CPC A61B 17/244; A61B 2017/00473

(57) ABSTRACT

The present disclosures present discloses novel and advantageous tongue-cleaner heads compatible, each compatible with a particular type of prevailing electric toothbrushes (such as Oral-B or Sonicare electric toothbrushes) and including a respective novel and advantageous tongue-cleaner unit having advantageous structures tailored or otherwise configured for one or more differentiated and advantageous tongue-cleaning actions. Such a tongue-cleaner head repurposes or otherwise transforms an otherwise host electric toothbrush into an advantageous electric tongue-cleaner after it is integrated with, the coupling handle of the otherwise host electric toothbrush. The resulting electric tongue cleaner is capable of performing advantageous and differentiated tongue-cleaning actions in its respective tongue-cleaning unit being mobilized by the coupling handle and undergoing a respective well-known distinct set of motions of one or more high frequencies that comes with the host electric toothbrush.

### CROSS-REFERENCE TO RELATED APPLICATION

This application is which is a non-provisional application claims the benefit under 35 U.S. C. § 119(e) of Provisional Ser. No. 63/686,845 , filed Aug. 25, 2024. The entire disclosure of this aforesaid prior applications is hereby incorporated by reference.

### RELATED ART

### 1. Technical Field

The present disclosure generally relates to a tongue-cleaning device and particularly relates to a tongue-cleaner head adapted for an attachment to a coupling handle of a prevailing electric toothbrush for turning the hosting electric toothbrush to an electrically powered tongue-cleaner.

### 2. Background

Electric toothbrushes are popular, widely available, and routinely used for tooth cleaning to the extent that they have already become staple oral hygiene items in people's daily lives. The two biggest and most popular brands are Oral-B of Procter and Gamble, and Sonicare of Phillips.

For ease of discussion, hereinafter, whenever a term such as “electric toothbrush,” “electric toothbrushes,” “toothbrush,” “toothbrushes,” or any of other similar terms, is used in a context in which the toothbrush or toothbrushes alluded to is of either the Oral-B brand or the Sonicare brand, the same refers to a typical electric toothbrush or typical electric toothbrushes of either the Oral-B brand or the Sonicare brand.

At the outset, electric toothbrushes of the Oral-B and Sonicare brands share one major commonality (hereinafter simply referred to as “the First Major Commonality”) well-known to the general public. Namely, an electric toothbrush of either brand includes a coupling handle and uses the coupling hand to power and mobilize a toothbrush head (hereinafter simply referred to as “brush head”) detachably attached and docked to the coupling handle to perform a teeth-brushing (i.e., teeth-cleaning) operation.

In more details with respect to the First Major Commonality shared by toothbrushes of both brands, for an electric toothbrush of either Oral-B or Sonicare, the coupling handle includes a housing where a set of electronic and mechanical components (for driving and controlling a brush head currently attached to the coupling handle) are housed. In a nutshell, this set of electronic and mechanical components collectively function as the “engine” and the “brain” of the electric toothbrush in terms of mobilizing and controlling the attached brush head for active teeth-cleaning (when the coupling handle is powered on and activated for active teeth-cleaning).

Further with respect to the First Major Commonality, for an electric toothbrush of either Oral-B or Sonicare, a compatible brush head attached to the coupling handle may be detached and removed therefrom, with which another compatible toothbrush head (serving as a replacement toothbrush head) may be detachably attached to the coupling handle for teeth-cleaning.

As used herein, “a brush head compatible to a certain brand” refers to a brush head specifically made and adapted to be attached and docked to a host electric toothbrush sold under one of a set of one or more corresponding models of that certain brand such as Oral-B or Sonicare, such that the brush head, upon having been—through its respective set of adapting attachment and docking interfaces (hereinafter collectively as a whole referred to as “the docking adapter” or “its docking adapter”) pre-provisioned inside or on its housing—attached and docked to the coupling handle of the host electric toothbrush, engages the coupling handle of the host electric toothbrush in a manner which makes the brush head to become, operation-wise, an integral part of the host electric toothbrush in that the brush head is under the full control of the coupling handle and, upon actuation (by, e.g. a user with the pressing of a “power-on” button), becomes mobilized in causing its transversely extending bristles (which will be further discussed below) to undergo movements (motions) in one or more pre-designed manners and fashions as intended by the coupling handle for the purpose of teeth-cleaning.

Still further with respect to the First Major Commonality, for an electric toothbrush of either Oral-B or Sonicare, the technical specifics of the respective docking adapter of, or otherwise needed to make, a compatible brush head is well-known to the extent that for electric toothbrushes of different models of both brands, third party vendors have been able to mass-produce and mass-sell compatible replacement brush heads to the general public.

Still further with respect to the First Major Commonality, a replacement brush head compatible for an electric toothbrush of either Oral-B or Sonicare—whether it be sold under either the Oral-B or Sonicare brand or under a third-party brand (i.e., a brand at large which is neither Oral-B nor Sonicare)—comprises a bristles-based brush unit at its distal end and an elongated shank extending longitudinally from the brush unit to its proximal end.

Hereinafter, the term “replacement brush head,” unless expressly prescribed to a specific brand, refers to a replacement brush head compatible for either the Oral-B brand or the Sonicare brand.

Still further with respect to the First Major Commonality, for a replacement brush head, its brush unit usually comprises a support base and multiple clusters or tufts of bristles extending transversely from the support base. Its elongated shank is, configured, on one hand, to securely attach the brush head to the coupling handle at its proximal end (which is also the proximal end of the brush head), and on the other hand, to receive in its housing a linkage component (e.g. in the form of a metal drive shaft or arm) of the coupling handle (a linkage component which extends longitudinally from the inside of the housing of the coupling handle to the outside thereof). The brush head is configured, via its docking adapter, to enable the linkage component to be coupled to the brush unit such that the brush unit, therefore the transversely extending bristles, can be mobilized through, inter alia, the linkage component and the docking adapter, by the set of electronic and mechanical components (which includes the linkage component) of the coupling handle in one or more specific manners intended and configured for teeth-cleaning.

Thus, the linkage component of the coupling handle may also be viewed as a drive component (i.e., a drive arm), which is instrumental in enabling the set of electronic and mechanical components of the coupling handle to mobilize the transversely extending bristles in one or more specific manners pre-devised and configured for teeth-cleaning.

Given the detachable nature of a brush head and the consideration that the coupling handle of an electric toothbrush of either Oral-B or Sonicare houses all remaining components of the electric toothbrush, including both the engine and the drive component thereof, the term “coupling handle” and the term “electric toothbrush,” when either is used in a context involving an electric toothbrush of either Oral-B or Sonicare, may be used herein interchangeably to refer to the coupling handle of an electric toothbrush (otherwise having a detachable brush head attached and docked to the coupling handle), regardless of whether a detachable brush head is actually attached and docked to the coupling handle or not.

Having discussed above the First Major Commonality shared between an electric toothbrush of Oral-B (hereinafter simply referred to as “an Oral-B toothbrush”) and an electric toothbrush of Sonicare (hereinafter simply referred to as “a Sonicare toothbrush”), there are also, at the outset, well-known distinctions existing between the two.

On one hand, for a Sonicare toothbrush, the brush unit of a compatible brush head is shaped generally elliptical, which is similar to the generally elliptical shape of the brush head of a regular traditional manual hand-held toothbrush. Further, in a nutshell, the entire brush unit of a Sonicare-compatible brush head, upon actuated for teeth-cleaning (through the brush head's docking adapter) by the coupling handle of a host Sonic toothbrush, vibrates and oscillates back and forth linearly at one or more high frequencies (e.g., at one or more acoustic or ultrasonic frequencies), resulting in the transversely extending bristles, as a part of the brush unit, vibrating and oscillating back and forth linearly in the same or a like manner at the same frequency.

In contrast, for an Oral-B toothbrush, the brush unit of a compatible brush head is shaped generally circular (in being adapted to make circular motions), which is unlike the generally elliptical shape of the brush head of a regular traditional non-electric toothbrush. In a nutshell, the support base of an Oral-B-compatible brush head (which is generally shaped circular), upon being actuated (through, inter alia, the brush head's docking adapter including its adapting component or components) by the coupling handle of a host Oral-B toothbrush for teeth-cleaning, rotates and oscillates, pulsates at one or more known frequencies (e.g., at one or more acoustic or ultrasonic frequencies), resulting in the transversely extending bristles rotating, oscillating and pulsating with the support base in the same or a similar manner at the same frequency.

These above-discussed distinctions (as relating to, for a replacement compatible brush head of either brand, inter alia, the general shape of the brush unit and the teeth-cleanings motions of the transversely extending bristles) between an Oral-B electric toothbrush and an Sonicare electric toothbrush, however, render both electric toothbrushes share another major commonality (hereinafter simply referred to as “the Second Major Commonality”). Namely, both electric toothbrushes have been widely acknowledged and accepted as producing superior and desirable teeth-cleaning results with these above-discussed respective distinctions of theirs.

Particularly, according to Sonicare (e.g., through its aired TV commercials), the generally linear back-and-forth motions of the bristles as a whole—when the tips thereof are positioned against, e.g., side surfaces of adjacent teeth and interdental regions there-between during a teeth-cleaning session—has proven to be advantageous for teeth-cleaning in producing superior and desirable teeth-cleaning results. Likewise, according to Oral-B (e.g., through its aired TV commercials), the rotatory and oscillatory motions of the transversely extending bristles (at an acoustic or ultrasonic frequency) as a whole—when, e.g., the tips thereof are positioned against, e.g., side surfaces of adjacent teeth and interdental regions there-between during a teeth-cleaning session—has proven to be advantageous for teeth-cleaning in producing superior and desirable tooth-cleaning results. Both Sonicare's and Oral-B's assertion of superior results are apparently invariably confirmed by the general public, as electric toothbrushes of both brands have been popular in having been purchased in mass quantities by the general public.

To summarize, for both Oral-B and Sonicare electric toothbrushes, they share the First and Second Major Commonalities. The First Major Commonality relates to the generally same modular approach employed and implemented by both brands. This modular approach, which separates the provisioning of a coupling handle from the provisioning of a detachably attachable and dockable brush head (with the docking adapter thereof) while enabling both to be integrally combined to perform the intended function of teeth-cleaning, has proven to be advantageous in the general field of teeth-cleaning. The Second Major Commonality relates to mobilizing the brush unit of a compatible brush head attached to the coupling handle, which results in the brush unit, and therefore the teeth-cleaning bristles, undergoing motions advantageous to teeth-cleaning. The Second Major Commonality, which is, as discussed above, widely confirmed by the general public, demonstrates that for both a Sonicare toothbrush and an Oral-B toothbrush, both respective distinct motions which the respective brush units of their respective brush heads (attached and docked to their respective coupling handles), and therefore their respective transversely extending teeth-cleaning bristles, undergo are advantageous to teeth-cleaning.

Turning to the general field of tongue-cleaning, in view of the above-discussed First and Second Major Commonalities shared between Oral-B and Sonicare electric brushes as well as the observation that both commonalities are advantageous to teeth-cleaning in producing superior and desirable teeth-cleaning results, innovators in the general field of tongue-cleaning have wondered whether and how the above-discussed First and Second Major Commonalities shared between Oral-B and Sonicare electric brushes may be leveraged to improve tongue-cleaning, which is distinct from teeth-cleaning in having distinct considerations as relating to the distinct characteristics and natures of a human tongue.

To the knowledge of the Inventor of the present disclosure, who is one of those innovators, attempts and products were indeed made to leverage electric toothbrushes of both Oral-B and Sonicare, particularly the above-discussed First and Second Major Commonalities shared between Oral-B and Sonicare electric brushes (which are advantageous to bristles-based teeth-cleaning), to improve tongue-cleaning. However, to the Inventor's knowledge and understanding, most of, if not all of, those past attempts and products were not adequately taking into both (a) the unique considerations and challenges that come with tongue-cleaning and (b) how, for electric toothbrushes of both Oral-B and Sonicare, the respective distinct motions, which the respective brush units of the respective brush heads are mobilized to undergo, may be effectively leveraged while without compromising, or otherwise working against, the unique considerations and challenges that come with tongue-cleaning.

### BRIEF SUMMARY

The present disclosure discloses novel and advantageous tongue-cleaner heads, each compatible with a particular type of prevailing electric toothbrushes (such as Oral-B or Sonicare electric toothbrushes) and including its respective novel and advantageous tongue-cleaner unit having advantageous structures extending upright from a surface thereof and tailored or otherwise configured for one or more differentiated and advantageous tongue-cleaning actions. Such a tongue-cleaner head, with its respective advantageous tongue-cleaner unit, repurposes or otherwise transforms an otherwise host electric toothbrush (with which it is compatible) into an advantageous electric tongue-cleaner after it is, through known docking interfaces, attached or docked to, or otherwise integrated with, the coupling handle of the otherwise host electric toothbrush. The resulting electric tongue cleaner is capable of performing advantageous and differentiated tongue-cleaning actions, resulting from the respective tongue-cleaner unit (of such a tongue-cleaner head), with its advantageous structures being tailored or otherwise configured for one or more differentiated and advantageous tongue-cleaning actions, being mobilized by the coupling handle and undergoing a respective well-known distinct set of motions of one or more high frequencies (otherwise intended for teeth-cleaning).

### BRIEF DESCRIPTION OF THE DRAWINGS

The description of the illustrative embodiments can be read in conjunction with the accompanying figures. It will be appreciated that for simplicity and clarity of illustration, elements illustrated in the figures, unless expressly specified, have not necessarily been drawn to scale. Also, any text and/or any numerical data (numbers) appeared on any drawing figures is provided to illustrate an exemplary embodiment or implementation, and thus is provided for the purpose of illustration and not for the purpose of limitation. For example, the dimensions of some of the elements may be exaggerated relative to other elements. Embodiments incorporating teachings of the present disclosure are shown and described with respect to the figures presented herein, in which:

FIGS. 1A-D are pictorials exemplifying Oral-B and Sonicare electric toothbrushes discussed above in connection with the related art.

FIGS. 2A-P are different views and diagrams illustrating an exemplary Oral-B-compatible tongue-cleaner unit 100 of an exemplary Oral-B-compatible tongue-cleaner head 130, in accordance with one or more embodiments of the present disclosure.

FIGS. 3A-J are different views illustrating an exemplary Oral-B-compatible tongue-cleaner unit 200 an exemplary Oral-B-compatible tongue-cleaner head 230, in accordance with one or more embodiments of the present disclosure.

FIGS. 4A-I are different views illustrating an exemplary Sonicare-compatible tongue-cleaner head 330, in accordance with one or more embodiments of the present disclosure.

FIGS. 5A-H are perspective views illustrating an exemplary mild type of Sonicare-compatible tongue-cleaner head 430, in accordance with one or more embodiments of the present disclosure.

### DETAILED DESCRIPTION

In the following detailed description of exemplary embodiments of the disclosure in this section, specific exemplary embodiments in which the disclosure may be practiced are described in sufficient detail to enable those skilled in the art to practice the disclosed embodiments. However, it is to be understood that the specific details presented need not be utilized to practice embodiments of the present disclosure. The following detailed description is, therefore, not to be taken in a limiting sense, and the scope of the present disclosure is defined by the appended claims and equivalents thereof.

In the description, relative terms such as “left,” “right,” “vertical,” “horizontal,” “upper,” “lower,” “front,” “back,” “side,” “upright,” “distal,” “proximal,” “top” and “bottom” as well as any derivatives thereof should be construed to refer to the logical orientation as then described or as shown in the drawing figure under discussion. Accordingly, these relative terms are for convenience of description and are not intended to convey any limitation with regard to a particular orientation.

Within the descriptions of the different views of the figures, the use of the same reference numerals and/or symbols in different drawing figures indicate similar or identical items, and similar and/or related elements may be referenced with similar names and reference numerals throughout the figures. If a reference numeral is once used to refer to a plurality of like elements, unless required otherwise by context, the reference numeral may refer to any, a subset of, or all of, the like elements in the figures bearing that reference numeral. A reference alpha-numeral (such as “101A”) may refer to one implementation, one subset, or one of several types, of one element or a plurality of like elements bearing the same base reference numeral (such as “101”). The specific identifiers/names and reference numerals assigned to elements (depicted in these figures) are provided solely to aid in the description and are not meant to imply any limitations (structural or functional or otherwise) on the described embodiments. Also, the depictions in these figures are for illustrative purposes only and are not drawn to scale.

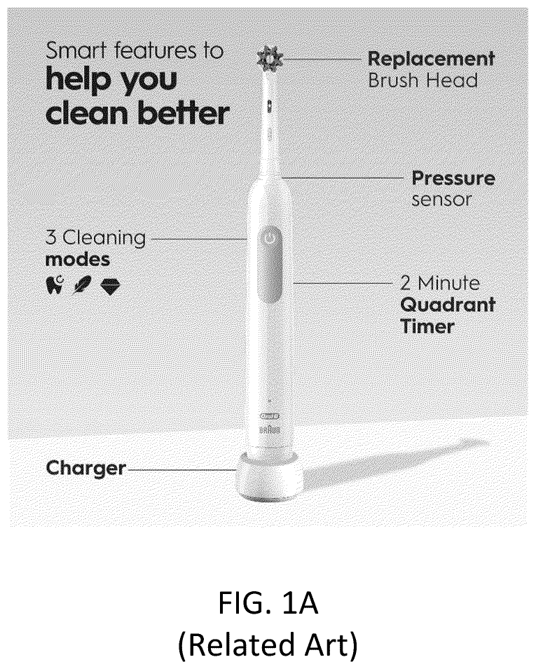

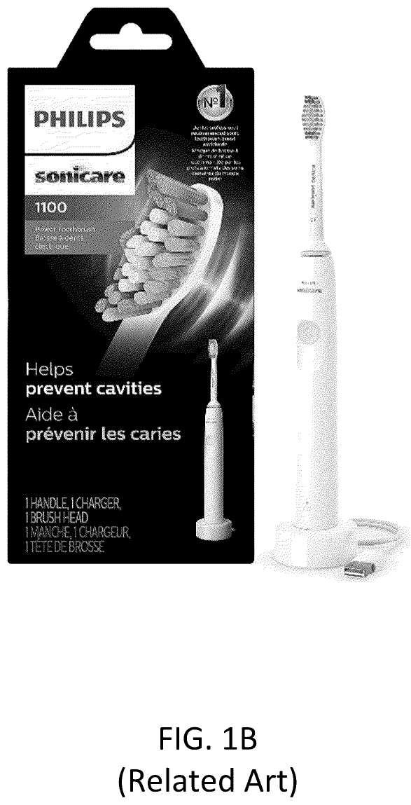

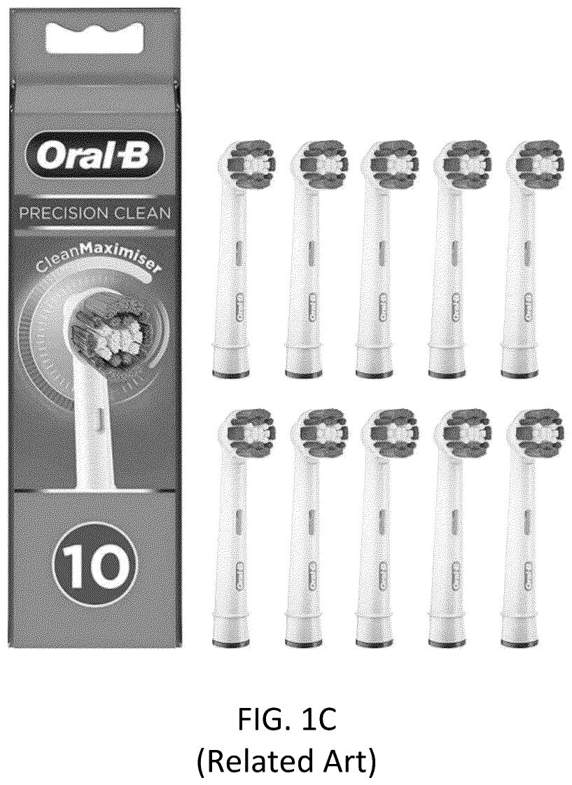

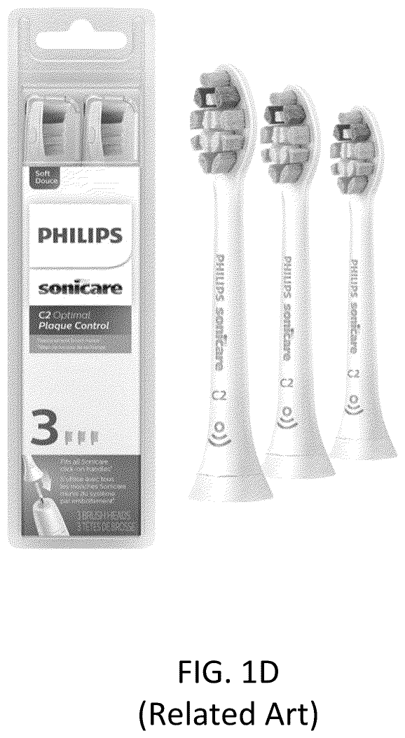

FIGS. 1A-D are pictorials exemplifying Oral-B and Sonicare electric toothbrushes discussed above in connection with the related art. More specifically, FIG. 1A shows an exemplary Oral-B electric toothbrush. FIG. 1B shows an exemplary Sonicare electric toothbrush. FIG. 1C shows examples of replacement brush heads compatible with Oral-B electric toothbrushes. FIG. 1D shows examples of replacement brush heads compatible with Sonicare electric toothbrushes.

Hereinafter, an “Oral-B-compatible tongue-cleaner unit” refers to a tongue-cleaner unit of the present disclosure, which is configured and adapted for substituting for the brush unit of an otherwise Oral-B-compatible host brush head and which, with the substitution, repurposes or otherwise transforms, the otherwise Oral-B-compatible host brush head into an advantageous tongue-cleaner head (i.e., a replacement head except for being adapted for tongue-cleaning actions) compatible with an Oral-B electric toothbrush. As a skilled artisan readily appreciates, an Oral-B-compatible tongue-cleaner unit retains or otherwise comprises, by definition, a set of interface elements of the docking adapter of the otherwise Oral-B-compatible host brush head as discussed above in connection with the related art, the set of interface elements which the substituted brush unit of an otherwise Oral-B-compatible host brush head comprises.

Hereinafter, an “Oral-B-compatible tongue-cleaner head” refers to an otherwise Oral-B-compatible brush head, which, with the substitution of an Oral-B-compatible tongue-cleaner unit for its typically circular shaped brush unit, is repurposed, or otherwise transformed, into an advantageous tongue-cleaner head compatible with an Oral-B electric toothbrush, in accordance with one or more embodiments of the present disclosure.

Hereinafter, an “Oral-B-repurposed tongue-cleaner” refers to an electric tongue-cleaner advantageously formed for performing tongue-cleaning actions, with the advantageous electric tongue-cleaner being an ordered combination of an Oral-B-compatible tongue-cleaner head and the coupling handle of an otherwise host Oral-B electric toothbrush, the ordered combination which is formed through attaching and docking the Oral-B-compatible tongue-cleaner head to the coupling handle through the docking adapter of the Oral-B-compatible tongue-cleaner head such that the Oral-B-compatible tongue-cleaner head, upon being docked, becomes an integral part of the ordered combination in being mobilized by the coupling handle and undergoing, as a result of the mobilization, rotatory, oscillating and pulsating motions in well-known distinct manners (otherwise intended for teeth-cleaning) for performing advantageous tongue-cleaning actions, in accordance with one or more embodiments of the present disclosure.

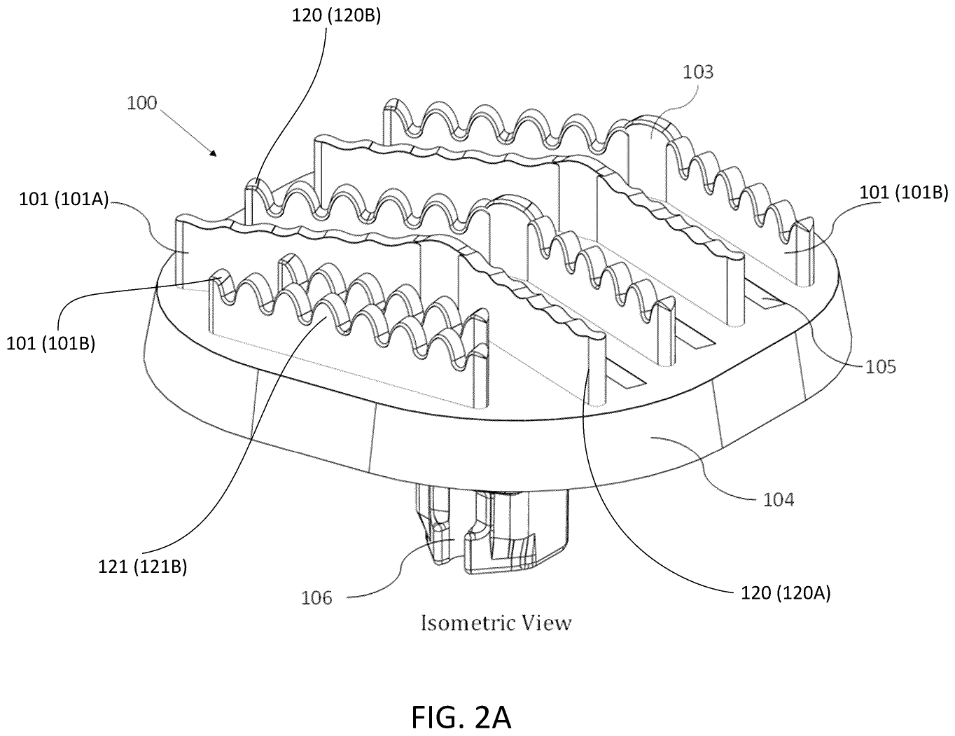

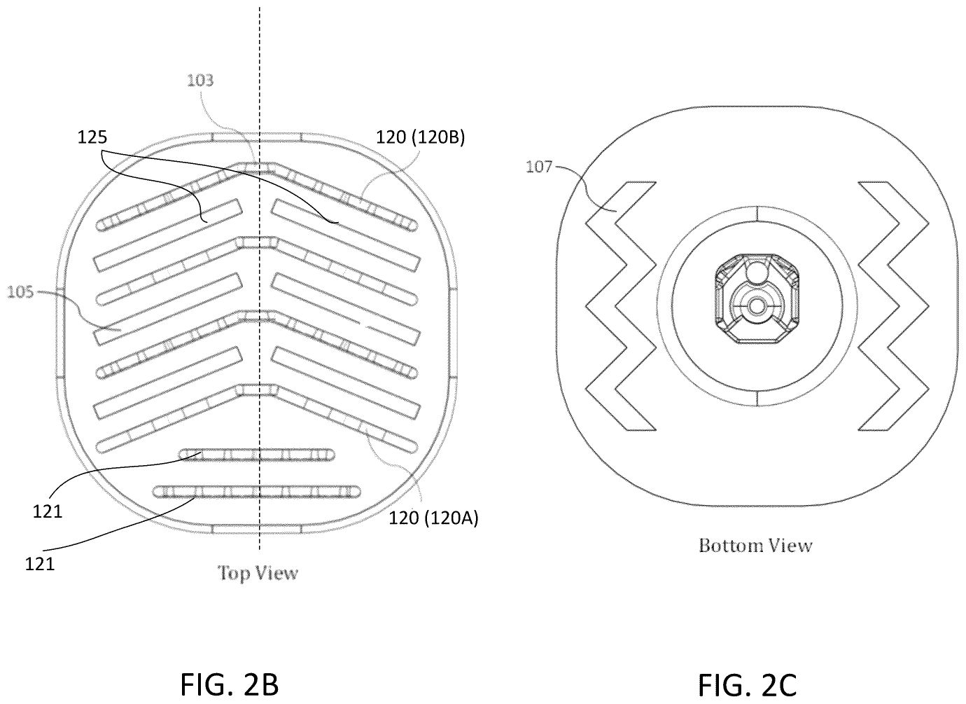

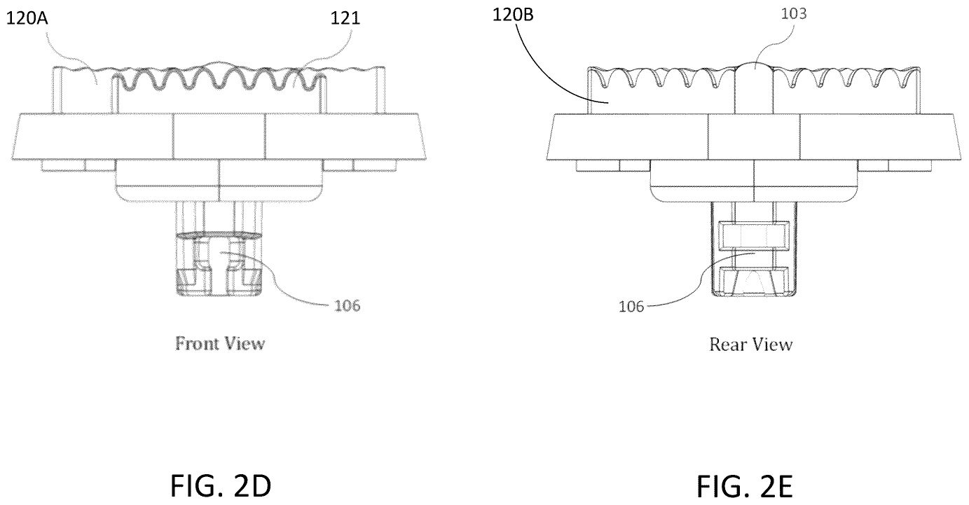

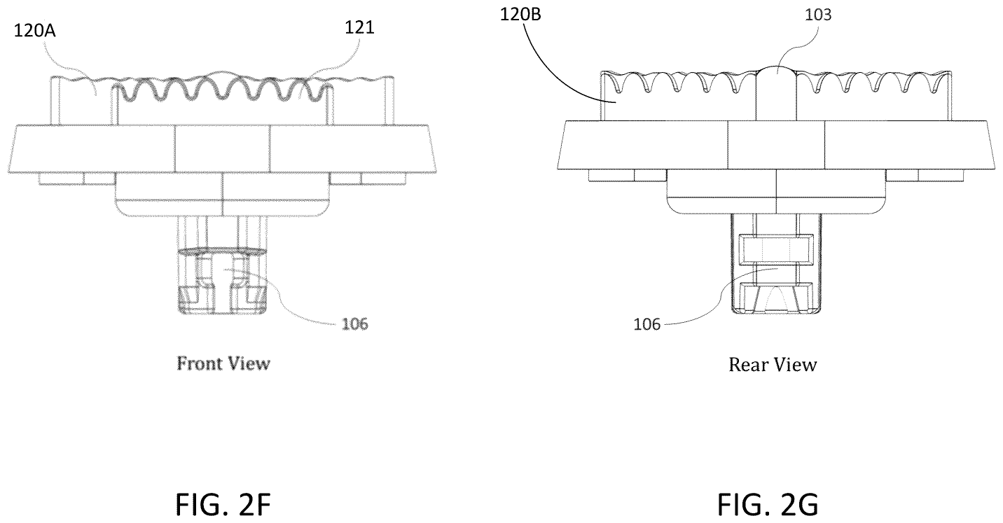

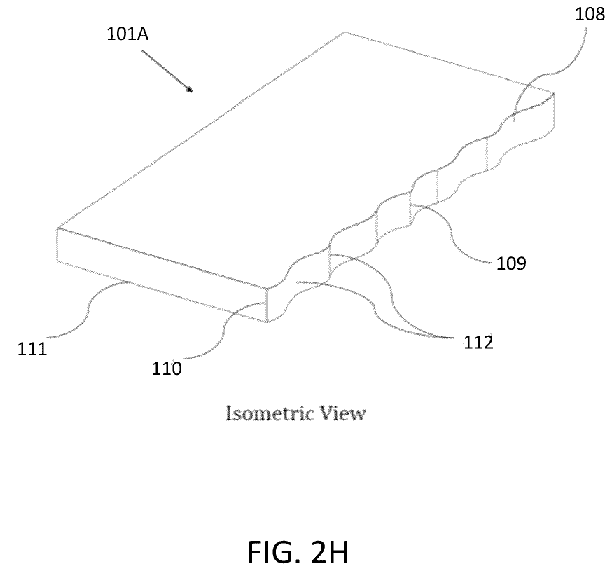

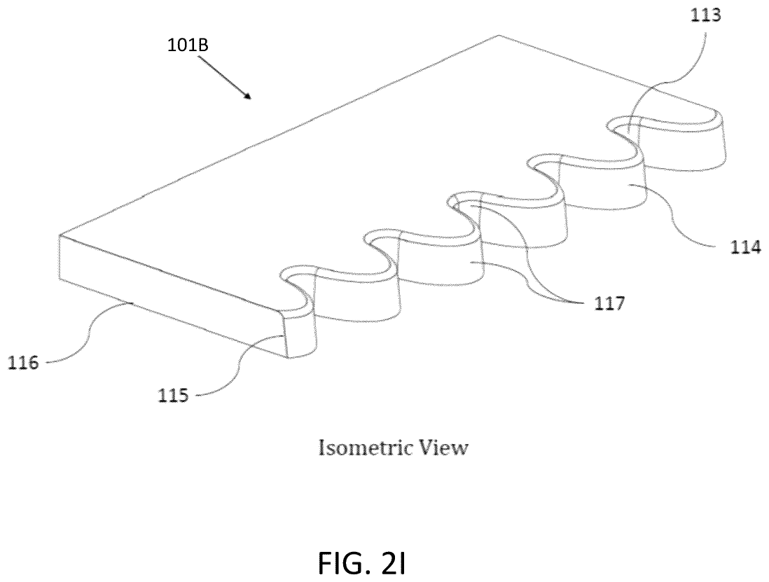

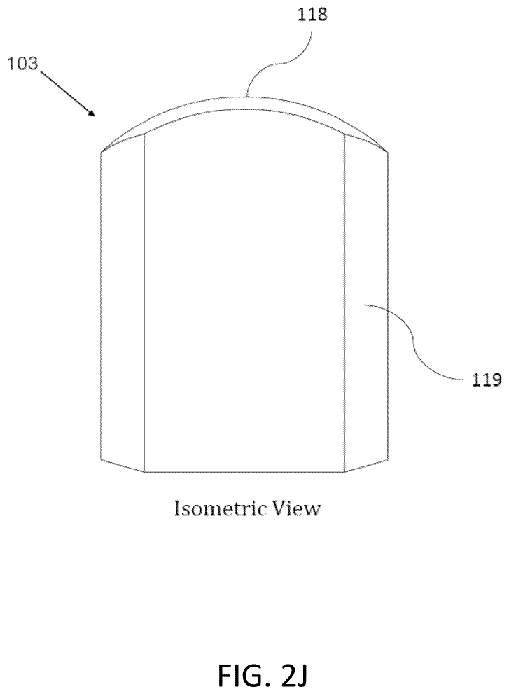

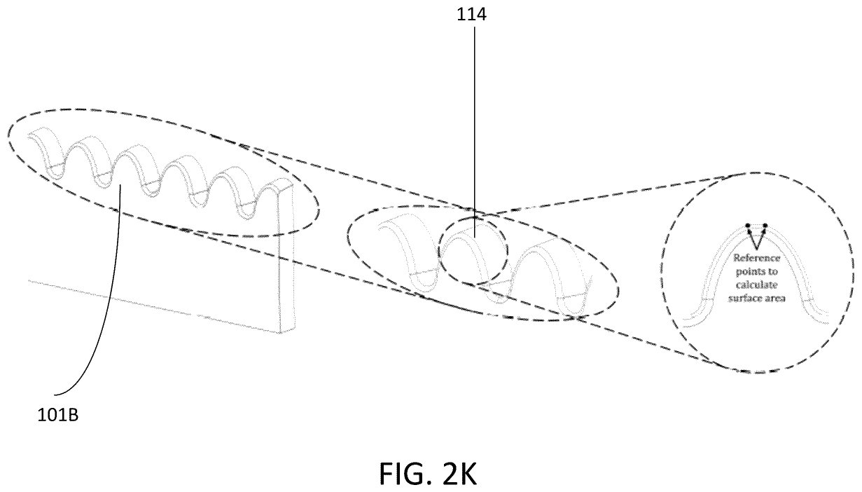

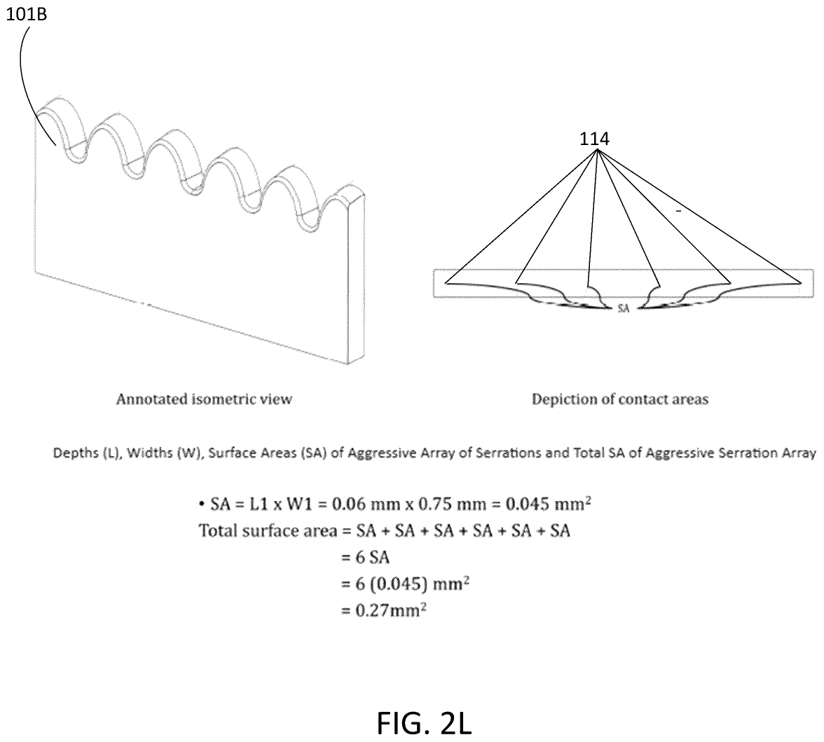

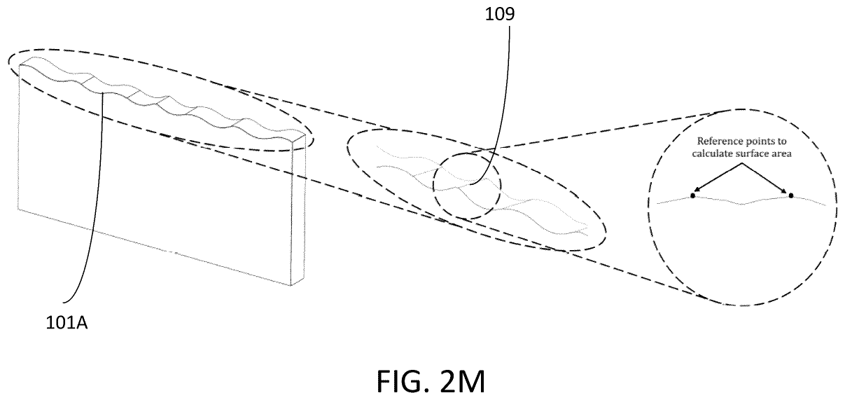

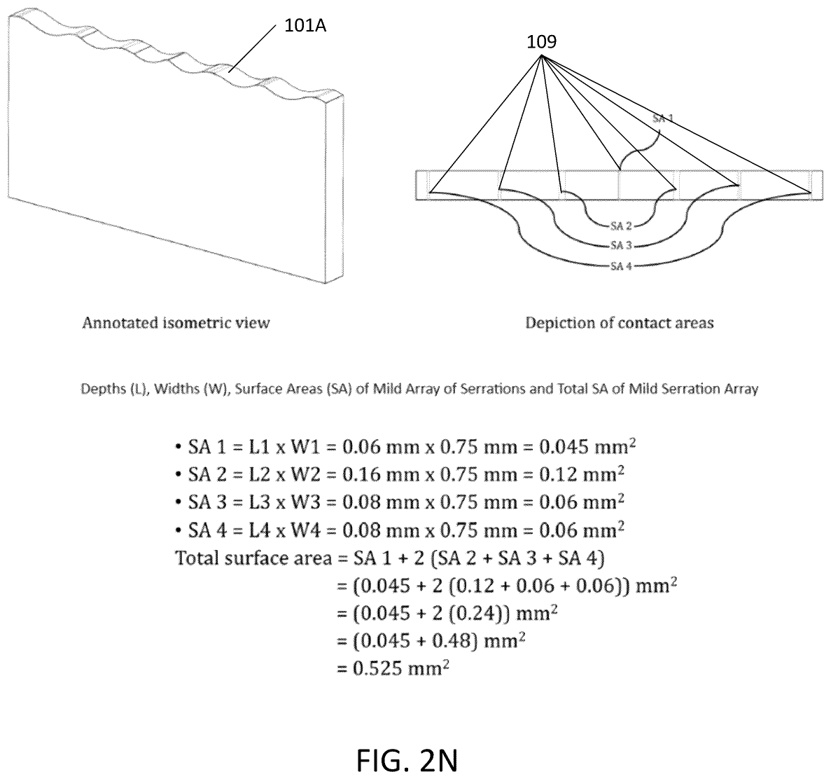

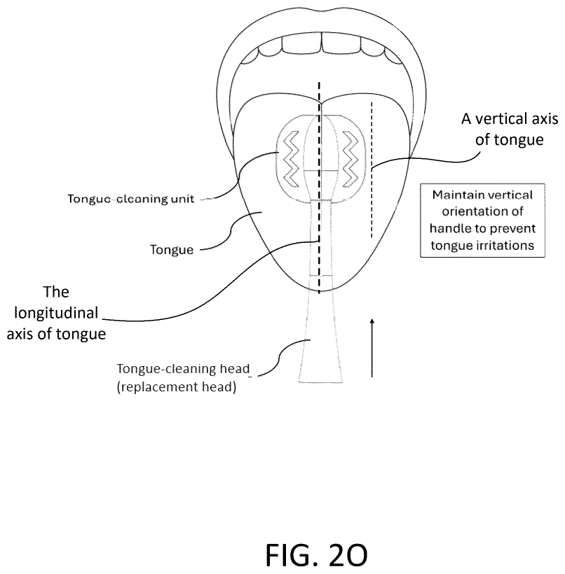

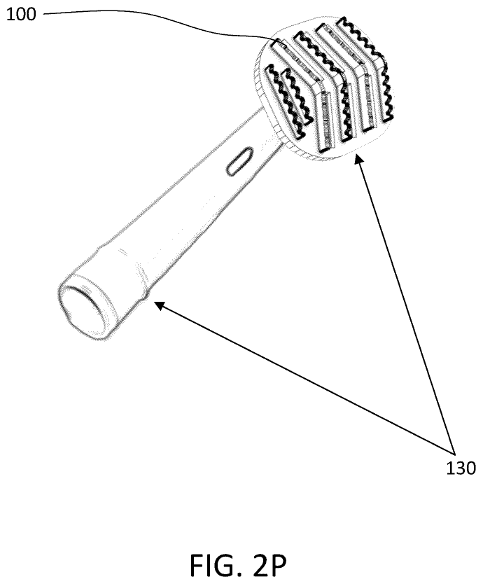

Hereinafter, “Oral-B-repurposed tongue-cleaning actions” refers to cleaning actions performed on the upper surface or surfaces of the body of a tongue using an Oral-B-repurposed tongue-cleaner, particularly a docked Oral-B-compatible tongue-cleaner unit thereof (in taking advantage of the rotatory, oscillating and pulsating motions which the docked Oral-B-compatible tongue-cleaner unit undergoes in well-known distinct manners when being mobilized by the coupling handle of the Oral-B-repurposed tongue-cleaner), with the docked Oral-B-compatible tongue-cleaner unit being placed against the upper surface or surfaces of the tongue body and properly oriented (i.e., oriented in one or more manners purported for tongue-cleaning), as is exemplified herein by FIG. 2O, in accordance with one or more embodiments of the present disclosure.

Hereinafter, an “Oral-B-repurposed tongue-cleaning operation” refers to a tongue-cleaning operation performed on the upper surface or surfaces of the body of a tongue using an Oral-B-repurposed tongue-cleaner, particularly the Oral-B-compatible tongue-cleaner unit thereof, an operation which, by definition, comprises Oral-B-repurposed tongue-cleaning actions.

FIGS. 2A-P are different views and diagrams illustrating an exemplary Oral-B-compatible tongue-cleaner unit 100 (hereinafter simply referred to as “the exemplary tongue-cleaner unit 100”) of an Oral-B-compatible tongue-cleaner head 130, which is configured to be of an aggressive type of an Oral-B-compatible tongue-cleaner unit.

An Oral-B-compatible tongue-cleaner unit 100 of an aggressive type (hereinafter simply referred to as “tongue-cleaner unit 100”) comprises an encompassing base 104. For the exemplary tongue-cleaner unit 100, its encompassing base 104, which is a support base, is configured, as shown, with draft angles, the provision of which stems from, inter alia, taking into account ease of manufacturability through injection molding.

A tongue-cleaner unit 100 comprises serration arrays deployed therein. Each serration array 101 are configured and adapted, e.g., through profiles of its respective serrations, for tongue-cleaning actions, such as scraping actions, of a certain level of hardness or softness, depending on the level of aggressiveness or mildness which the respective serrations as a whole is considered. Thus, a tongue-cleaner unit 100 may comprise serration arrays deployed therein configured and adapted for different and varying levels of hardness or softness in nature with regard to their respective tongue-cleaning actions.

As illustrated, the exemplary tongue-cleaner unit 100 comprises serration arrays 101A configured to be mild (in nature or level of aggressiveness or mildness) and serration arrays 101B configured to be aggressive (in nature or level of aggressiveness or mildness). Aggressive serration arrays 101B are configured and adapted for, e.g., scraping actions of being relatively hard in nature or level of hardness or softness. Mild serration arrays 101A are configured and adapted for, e.g., scraping actions of being relatively soft in nature or level. Putting it differently, mild serration arrays 101A are configured and adapted for, e.g., scraping actions of softer, or alternatively speaking less hard, in nature or level than that for aggressive serration arrays 101B.

As used herein in connection with a serration, a serration array, or a serration row, the apex surface or surfaces of a structure disposed upright (whether the structure be, e.g., a serration, a serration array, or a serration row) refers to, as applicable, one or more substantially or approximately flat surfaces of the upright structure disposed at substantially the highest vertical level of the upright structure. As used herein in connection with a serration, a serration array or a serration row, the apex or apexes of a structure disposed upright (whether the structure be, e.g., a serration, a serration array, a serration row) refers to, as applicable, one or more areas of the upright structure, regardless of the respective shape or contour of each of the one or more areas and how narrow, thin, or otherwise small, each of the one or more areas is, disposed at substantially the highest vertical level of the upright structure.

For a serration array (such as a serration array 101), its level of aggressiveness or mildness may be determined after the configurations of the individual serrations of the serration array are considered as a whole. A serration array becomes—among or otherwise besides other considerations, such as, e.g., the depths (thicknesses), the widths, or the surface areas of the respective apexes of the respective serration array, as exemplified and demonstrated in FIGS. 2K, 2L, 2M and 2N—more aggressive (i.e., less mild) when the apexes of its serrations are uniformly or mostly higher, and the troughs (valleys) of its serrations are uniformly or mostly deeper. For an individual serration of a serration array, an increase in the vertical height difference between the maxima and the minima of its respective serration profile results in an increase in level of aggressiveness, or alternatively speaking, a decrease in level of mildness. Conversely, the serration array becomes milder (i.e., less aggressive) in nature when the apexes of its serrations are uniformly or mostly shorter, and the troughs (valleys) of its serrations are uniformly or mostly shallower. For an individual serration of a serration array 101, a decrease in the vertical height difference between the maxima and the minima of its respective serration profile results in an increase in level of mildness, or alternatively speaking, a decrease in level of aggressiveness. FIGS. 2A-2N illustrate both exemplary mild serration arrays 101A (i.e., serration arrays configured to be, or otherwise considered, “mild”) and exemplary aggressive serration arrays 101B (i.e., serration arrays configured to be, or otherwise considered, “aggressive”), as serration arrays of both these two distinct types are deployed in an Oral-B-compatible tongue-cleaner unit 100 (which is configured to be, and considered, an aggressive type of Oral-B-compatible tongue-cleaner unit).

As used herein, the projecting height of a serration of a serration array of an Oral-B-compatible tongue-cleaner unit refers to the vertical distance, as measured from the top surface of the encompassing base thereof where the serration array meets the encompassing base to the highest point of the apex surface of the serration.

As used herein, the projecting height of a serration array of an Oral-B-compatible tongue-cleaner unit refers to the maximum height among the respective projecting heights of the serrations of the serration array.

Referring to FIG. 2H, which provides an example of a mild serration array 101A, it is formed of one or more exemplary troughs (valleys) 108 and one or more exemplary apexes 109, with serration depths 112 therebetween. In one embodiment, an exemplary serration depth 112 is 0.3 mm or therearound. A mild serration array 101A may be configured to have a smooth surface (whose edge slope 110 may be, as exemplified, around zero degree) adjacent to an array profile edge 111 (i.e., an serration array profile edge 111). In one embodiment, the height of an array profile edge 111 is 5 mm or therearound, of which 3 mm is above encompassing base 104 after the mild serration array is deployed onto encompassing base 104.

Referring to FIG. 2I, which provides an example of an aggressive serration array 101B, it is formed of one or more exemplary troughs (valleys) 113 and one or more exemplary apexes 114, with serration depths 117 therebetween. In one embodiment, an exemplary serration depth 117 is 1.35 mm or therearound. An aggressive serration array 101B may be configured to have a smooth surface (whose edge slope 115 may be, as exemplified, around five (5) degrees) extending from and adjacent to an array profile edge 116 (i.e., a serration array profile edge 116). In one embodiment, the height of profile edge 116 is, same as that of an exemplary mild serration array 101A, also 5 mm or therearound, of which 3 mm is above encompassing base 104 after the exemplary aggressive serration array 101B is deployed onto encompassing base 104.

Further referring to FIGS. 2K, 2L, 2M and 2N, as exemplified and demonstrated, for a serration array 101, configurations of the respective apexes of the serration array's respective serrations (such as the respective depths, widths, as well as surface areas of its respective serrations), are, when considered as a whole, revealing and determinative of, if not dispositive of, the serration array's nature or level of aggressiveness or, alternatively speaking, mildness.

A tongue-cleaner unit 100 may comprise one or more bridges 103 between adjacent serration arrays. A bridge 103 is of a profile configured to eliminate, or otherwise smooth out, the edge corners of adjacent serration arrays, which otherwise may irritate a tongue (during an Oral-B-repurposed tongue-cleaning operation).

In the exemplary tongue-cleaner unit 100, one bridge 103 is provided between two adjacent mild serration arrays 101A. Similarly, another bridge 103 provided between two adjacent aggressive serration arrays 101B. In this example, each bridge 103 is also joined, through the two respective angled faces 119 thereof, to both respective adjacent serration arrays 101.

A tongue-cleaner unit 100 may be configured to have one or more serration rows 120 each formed of, inter alia, one or more serration arrays 101. In this respect, the exemplary tongue-cleaner unit 100 is configured to have a plurality of serration rows 120 each formed of, inter alia, two serration arrays of a same level, or like levels, of aggressiveness or mildness. For an exemplary tongue-cleaner unit 100, it is configured to have a plurality of serration rows 120A each formed of, inter alia, two serration arrays 101A, both of which are of a same level, or like levels, of aggressiveness considered mild. The exemplary tongue-cleaner unit 100 is configured as well to have a plurality of serration rows 120B each formed of, inter alia, serration arrays of a same level, or like levels, of aggressiveness considered aggressive.

For each of serration rows 120A and 120B, the respective serration row forms a tongue-cleaning instrument row configured to perform respective cleaning actions of one or more distinct natures or levels. In one example, a respective serration row 120A is configured, with its respective serration arrays configured to be mild (in nature or level), to perform tongue-cleaning actions (e.g., scraping actions) that are relatively soft in nature or level. A respective serration row 120B is configured, with its respective serration arrays configured to be aggressive, to perform tongue-cleaning actions (e.g., scraping actions) that are relatively hard in nature or level. In one embodiment, as illustrated, serration rows 120A and 120B are alternatingly deployed, or otherwise alternatingly provided, transversely along one or more longitudinal axes 128 of the exemplary tongue-cleaner unit 100.

For a tongue-cleaning operation, such an alternating configuration, or any configuration similar thereto, is advantageous. Such an alternating configuration results in, inter alia, surface areas of a tongue receiving, from a tongue-cleaner unit 100, cleaning actions (including, e.g., scraping actions) of alternating levels of hardness or softness in very rapid successions commensurate with, e.g., the frequency or frequencies in which the tongue-cleaner unit 100 is mobilized to rotate, oscillate and pulsate during an Oral-B-repurposed tongue-cleaning operation. A tongue-cleaner unit 100 (which is of an aggressive type) with such an alternating configuration is believed to lead to, from the perspective of a user, a better overall user experience when taking into account both cleaning experience and cleaning results, compared to a tongue-cleaner unit of an aggressive type with, e.g., a rather homogenized configuration (with regard to deployment of multiple serration rows each configured and adapted to perform, e.g., scraping actions, of a respective nature or level in hardness or softness).

With regard to serration rows 120, as shown, for each serration row 120 deployed on the exemplary tongue-cleaner unit 100, in one aspect relating to angling, its two respective serration arrays 101 (which are joined to a same respective bridge 103 at two respective sides thereof) are both configured to have their respective lateral-extending axes form respective angles with respect to a longitudinal axis 128 of the exemplary tongue-cleaner unit 100. In one embodiment, the longitudinal axis 128 of the exemplary tongue-cleaner unit 100 roughly coincides with longitudinal axis of the body of a tongue—which extends longitudinally from the apex (proximal tip) of the tongue towards, e.g., the distal end of the midline groove of the tongue, as shown in FIG. 2O—when an Oral-B-compatible tongue-cleaner head (having an exemplary tongue-cleaner unit 100) is, as exemplified in FIG. 2O, properly aligned, or otherwise placed, on the tongue in its purported orientation for an Oral-B-repurposed tongue-cleaning operation.

Hereinafter, this longitudinal axis of the body of a tongue will be simply referred to as “the vertical axis” of a tongue or of the body of a tongue, as illustrated in FIG. 2O. Any longitudinal axis of the body of a tongue extending from one proximal part of the body of the tongue to one distal part thereof, an axis which is by definition parallel to the vertical axis of the tongue, will be referred to as “a vertical axis” of a tongue or of the body of a tongue, as illustrated in FIG. 2O.

This angling aspect (as relating to the two respective serration arrays 101 of each serration row 120), is provided, and configured, to adapt to known specifics relating to one or more angles of rotation which the otherwise brush unit of an Oral-B-compatible brush head undergoes at a high frequency (when the brush unit is being mobilized during an otherwise teeth-cleaning operation performed by a host Oral-B electric toothbrush). As one example, Oral-B electric toothbrushes of Oral-B's model 1000 are known to mobilize the brush unit (which is usually round-shaped) of an Oral-B-compatible brush head in distinct manners, manners which include alternately rotating the brush unit +45-degree (e.g., rotating 45-degree in one direction such as clockwise) and −45 degree (e.g., rotating back 45-dgreee in the opposing direction such as counter-clockwise if the one direction is clockwise) in succession at one or more known high frequencies (such as one or more acoustic or ultrasonic frequencies) such that the brush unit undergoes oscillating movements of back-and-forth in two opposite rotation directions in rapid succession.

Given these specifics (of, e.g., Oral-B's model 1000 electric toothbrushes) relating to rotation angles (i.e., specifics relating to the exemplary +45 degree and −45-degree rotation angles discussed above), for each serration row 120 deployed on an exemplary tongue-cleaner unit 100 (provided to be compatible with, e.g., Oral-B's model 1000 electric toothbrushes), its two respective serration arrays 101 (which are both joined to a respective bridge 103) are both configured to have their respective lateral-extending axes form such respective angles with respect to the longitudinal axis 128 of the exemplary tongue-cleaner unit 100 that for each of the two respective serration arrays 101, during an Oral-B-repurposed tongue-cleaning operation (using the exemplary tongue-cleaner unit 100) where the exemplary tongue-cleaner unit 100 is being mobilized to undergo rotating in two opposite rotation directions in succession at one or more known high frequencies while the exemplary tongue-cleaner unit 100 is properly aligned, or otherwise placed, on the tongue in its purported orientation, the rotation of the respective serration array in either of the two opposite directions does not render its lateral-extending axis ever getting aligned, or ever getting close to be aligned, with a vertical axis of the tongue.

That is, given a known specific degree (e.g., a 45-degree) of rotation in two alternating and opposite directions for the brush unit of an Oral-B-compatible brush head, for each serration row 120 of an exemplary tongue-cleaner unit 100, its two respective serration arrays 101 are both adapted to, and configured for, that known specific degree of rotation in two alternating and opposite directions, in that for each of its two respective serration arrays 101, the rotation of the respective serration array in both opposite directions is limited to a rotation range within which the lateral-extending axis of the respective serration array does not ever get aligned, or ever gets close to be aligned, along a vertical axis of the tongue.

As a skilled artisan appreciates, with this angling aspect (as being provided to the two respective serration arrays 101 of each serration row 120), scenarios surrounding tongue-cleaning, e.g., scraping actions where a serration array becomes aligned with a vertical axis of the tongue, which may potentially cause irritation to the tongue, is eliminated during an Oral-B-repurposed tongue-cleaning operation (during which the exemplary tongue-cleaner unit 100 is properly oriented). That is, the provision of this angling aspect prevents any sort of irritation caused to the tongue, which is advantageous for an Oral-B-compatible tongue-cleaner unit 100 of an aggressive type.

A tongue-cleaner unit 100 may comprise one or more debris collection channels 105 in, e.g., one or more areas where loose debris (generated during an Oral-B-repurposed tongue-cleaning operation) is expected to be visible. Such a debris collection channel 105, with debris collected therein, is instrumental in providing to a user a visual feedback of tongue-cleaning actions already performed, which is important for the experience of the user (in performing an Oral-B-repurposed tongue-cleaning operation using the tongue-cleaner unit 100) as it assists in viewing results and assessing a level of effectiveness of the on-going tongue-cleaning operation from the user's perspective. Thus, each debris collection channel 105, which may be implemented in a form of a recessed receptacle, is also a tongue-cleaning instrument despite the fact that it does not perform, e.g., removing unwanted solid articles off a tongue, directly during an Oral-B-repurposed tongue-cleaning operation. As illustrated, for the exemplary tongue-cleaner unit 100, debris collection channels 105 are deployed in rows 125 adjacent to a serration row 120 and in between two serration rows, such as serration rows 120A and 120B. Thus, similar to a serration row 120, a row 125 of debris collection channels 105, which is in between two serration rows 120, is also an instrument row.

A tongue-cleaner unit 100 may, if space permits on encompassing base 104 after deployment of serration rows 120, further comprise one or more serration rows 121B each comprising one single aggressive serration array 101B oriented horizontal relative to a longitudinal axis 128 (i.e., an axis orthogonal to a horizontally oriented serration row 121B) of the exemplary tongue-cleaner unit 100 and deployed adjacent to a portion of the perimeter of the tongue-cleaner unit 100 as the last one or two instrument rows transversely lined along a longitudinal axis 128. The deployment, or otherwise provision, of the one or more serration rows 121B on encompassing base 104 adds, or otherwise provides, another stage, or otherwise another variation, in terms of tongue-cleaning actions during an Oral-B-repurposed tongue-cleaning operation. In the case of an exemplary tongue-cleaner unit 100, two such serration rows 121B, in taking advantage of availability of areas remaining in encompassing base 104, are deployed as the last two horizontally deployed instrument rows lined along the longitudinal axis 128.

The exemplary tongue-cleaner unit 100 may comprise a symbol 107 that signifies its aggressive nature. Symbol 107 may use a zig-zag pattern, as exemplified, to indicate the notion of being aggressive in general or a certain level of aggressiveness as corresponding to, or reflective of, the exemplary tongue-cleaner unit 100 with regard to its serrations deployed thereon as a whole.

A tongue-cleaner unit 100 comprises, as illustrated by an exemplary tongue-cleaner unit 100, a set of one or more interface elements 106, which is an integral part of the respective docking adapter of an Oral-B-compatible tongue-cleaner head (hosting the exemplary tongue-cleaner unit 100) provided, as discussed above, to attach and dock the host Oral-B-compatible tongue-cleaner head (as if the host Oral-B-compatible tongue-cleaner head is an Oral-B-compatible replacement brush head) to the coupling handle of a host Oral-B electric toothbrush. As discussed above, technical details of respective docking adapters of respective replacement heads compatible for different or otherwise various models of Oral-B electric toothbrushes (i.e., compatible for respective coupling handles of different models of Oral-B electric toothbrushes) are well known in the related art. Thus, the set of one or more exemplary interface elements 106 are provided herein for the purpose of illustrating and not for the purpose of limiting the scope of the present disclosure.

As exemplified, the set of one or more interface elements 106 are provided to interface between an exemplary tongue-cleaner unit 100 and, e.g., a drive shaft of the respective coupling handle of an Oral-B electric toothbrush—the drive shaft is, as well known, provided as a part of a respective coupling handle, and configured to engage an Oral-B-compatible brush head (i.e., a replacement brush head)—in mobilizing the exemplary tongue-cleaner unit 100 to undergo well-known respective rotatory (i.e., radial), oscillating and pulsating teeth-cleaning actions at one or more known high frequencies (such as one or more acoustic or ultrasonic frequencies) through, inter alia, delivering power via the respective well-known electro-mechanical system (including the drive shaft) of, and provided inside the housing of, the respective coupling handle.

In one embodiment, as exemplified, for each serration array 101 of a tongue-cleaner unit 100, after being deployed onto encompassing 104, the projecting height of the respective serration array is configured to be 3 mm or therearound.

As used herein, the projecting height of a bridge 103 of an Oral-B-compatible tongue-cleaner unit refers to the vertical distance, as measured from the surface of the encompassing base 104 thereof where the bridge meets the encompassing base to the apex of the bridge 103.

In one embodiment, the respective projecting height of each bridge 103, after being deployed onto encompassing base 104, is configured to be 3.65 mm or therearound. This configuration entails that, the non-serrated apex surface 118 of a bridge 103, which is configured in a manner to prevent or otherwise reduce irritation of a tongue during an Oral-B-repurposed tongue-cleaning operation, is taller than the adjacent serration arrays 101 in a same respective serration row 120. This results in apex surface 118 of the respective bridge 103, rather than serrations of the two respective adjacent serration arrays 101, coming in contact with the tongue when the exemplary tongue-cleaner unit 100 is placed, or otherwise deployed, on the tongue while idle during the tongue-cleaning operation.

In one embodiment, as exemplified, for an exemplary tongue-cleaner unit 100, the respective longitudinal distances between two longitudinally adjacent serration rows 120 (as, e.g., measured between two longitudinally adjacent bridges 103), the respective longitudinal distance between the two horizontally deployed serration rows 121B, as well as the respective longitudinal distance between the horizontally deployed serration row 121B (longitudinally adjacent to a serration row 120) and its longitudinally adjacent serration row 120 (as, e.g., measured between the serration row 121B and the respective bridge 103 of the adjacent serration row 120), are uniformly configured to be 5 mm or therearound.

In one embodiment, for each serration row 120 of an exemplary tongue-cleaner unit 100, the respective angles between the respective lateral-extending axes of the two respective serration arrays 101 subtended at the respective bridge 103, are uniformly 135 degrees or therearound.

In one embodiment, for an exemplary tongue-cleaner unit 100, each of its debris-collection channels 105 is uniformly configured to have a respective recessed receptable configured, in having a respective depth deep enough to collect sufficient debris and provide visual feedback, to have a depth of 1.5 mm or therearound.

In one embodiment, for an exemplary tongue-cleaner unit 100, its encompassing base 104 is configured to be 2 mm or therearound in upright thickness. Combining with the exemplary 3 mm (or therearound) projecting height of a serration array 101, this exemplary 2 mm (or therearound) upright thickness of encompassing base 104 makes the total displacement height of a serration array 101, as measured from the bottom of its encompassing base 104 to the apex or apexes of the serration array 101, to be 5 mm or therearound.

In one embodiment, for the exemplary tongue-cleaner unit 100, its encompassing base 104 is configured to be circular or quasi-circular in shape. For its circular encompassing base 104, having a too-small diameter may result in a user being forced to use a higher-than-desirable number of strokes of tongue-cleaning actions during an Oral-B-repurposed tongue-cleaning operation, whereas having a too-big diameter may result in a higher-than-desirable likelihood of the encompassing base 104 colliding with other body parts inside the mouth of a user during an Oral-B-repurposed tongue-cleaning operation. Balancing these two considerations, the circular or quasi-circular encompassing base 104 is configured to have a diameter or quasi-diameter roughly being two times that of the circular brush unit of a commercially available Oral-B toothbrush head.

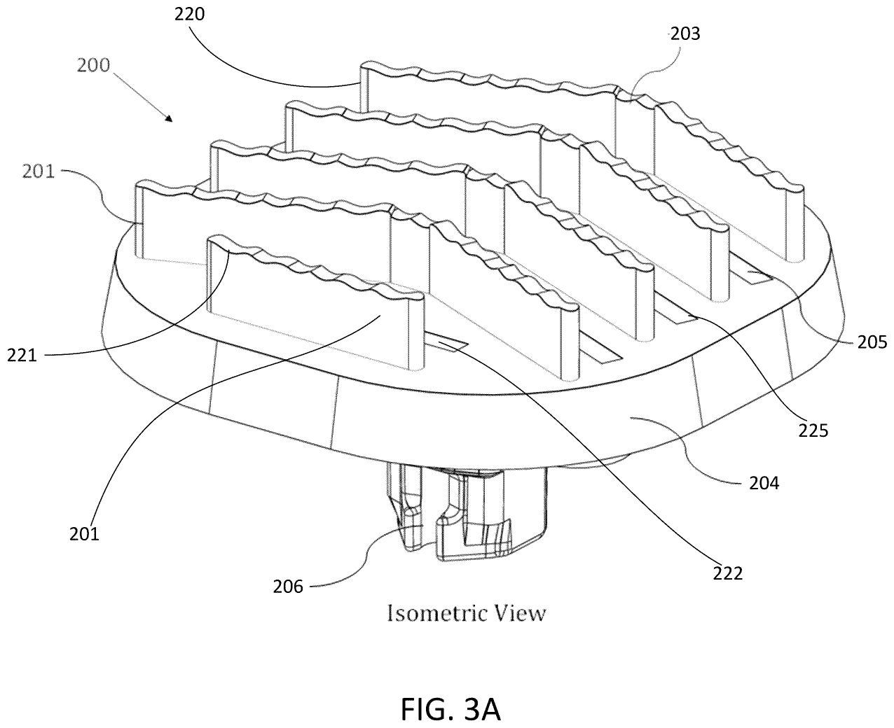

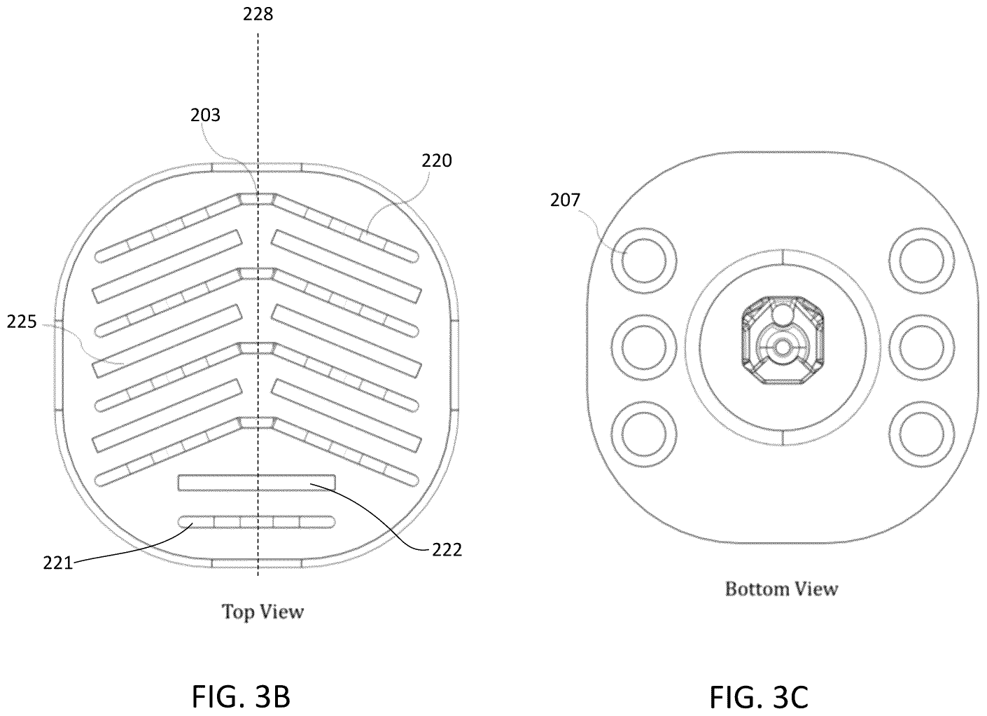

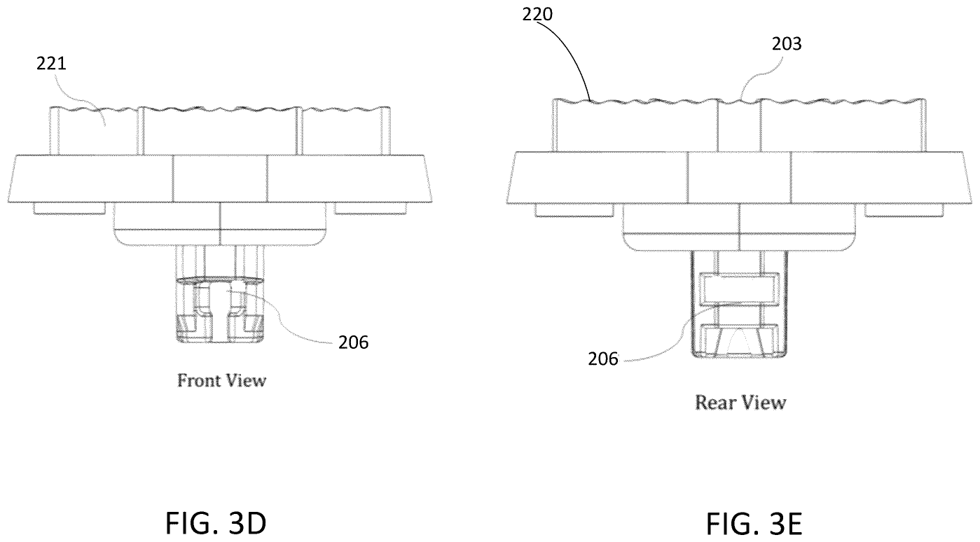

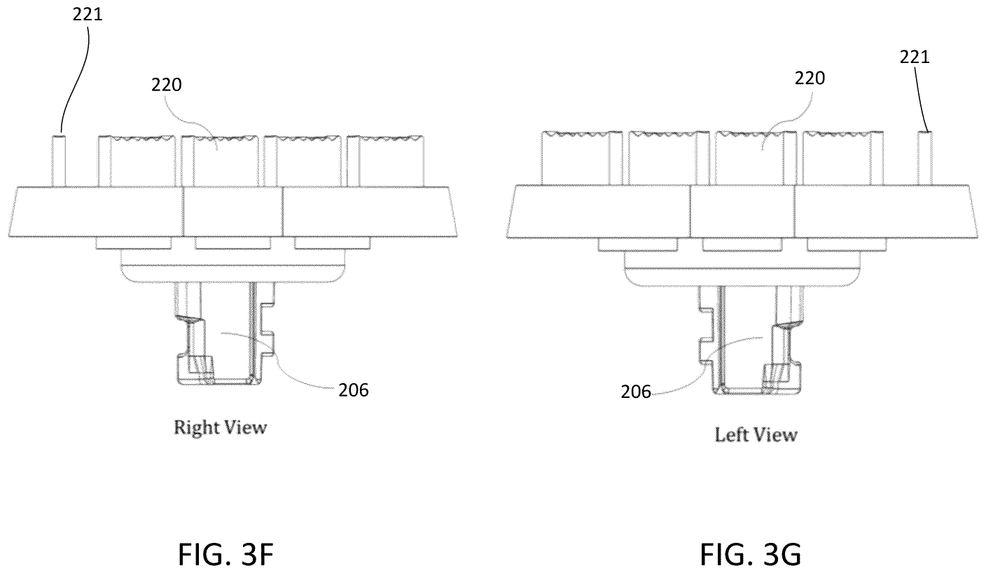

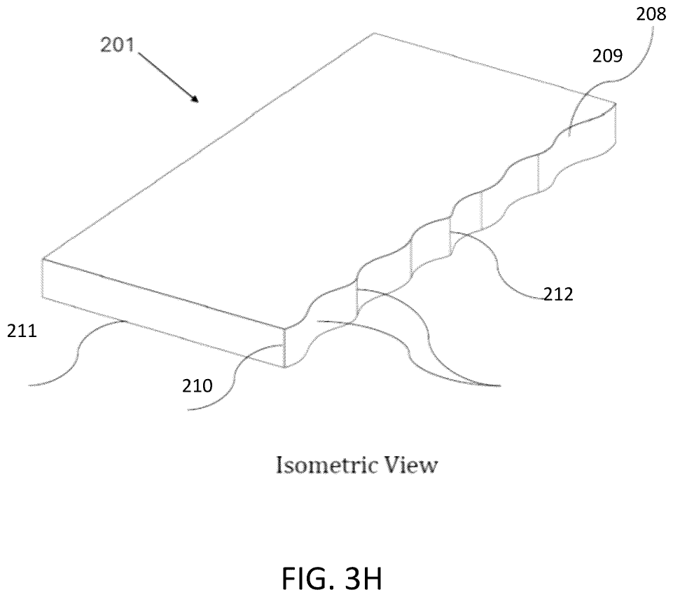

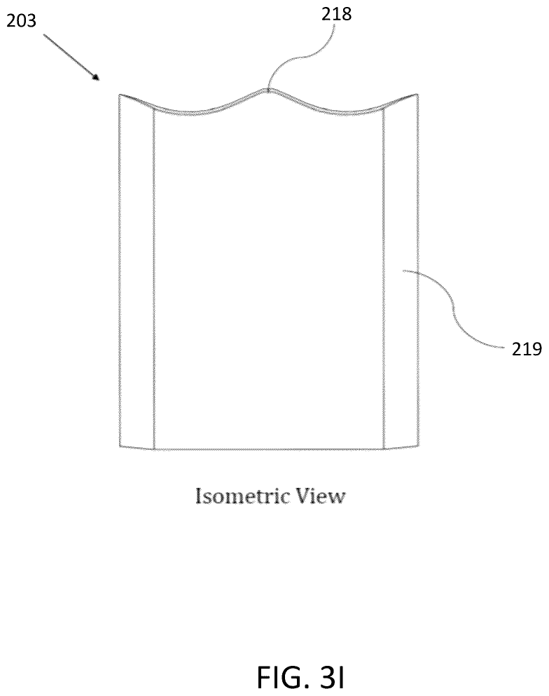

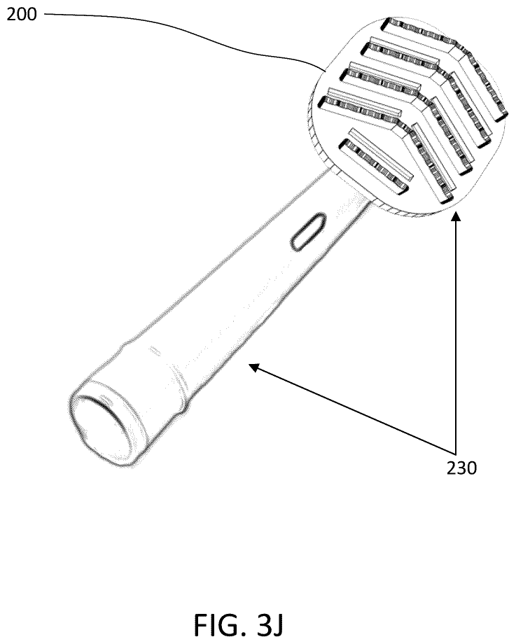

FIGS. 3A-J are different views illustrating an exemplary Oral-B-compatible tongue-cleaner unit 200 (hereinafter simply referred to as “exemplary tongue-cleaner unit 200”) of an exemplary Oral-B-compatible tongue-cleaner head 230, which is configured to be of a mild type of Oral-B-compatible tongue-cleaner unit. Hereinafter, an Oral-B-compatible tongue-cleaner unit of a mild type will simply be referred to a “tongue-cleaner unit 200.”

Similar to a tongue-cleaner unit 100, a tongue-cleaner unit 200 comprises a set of one or more interface elements 206, which, in one embodiment, is a same set of one or more interface elements 106 of a tongue-cleaner unit 100, which is an integral part of the respective docking adapter of an Oral-B-compatible tongue-cleaner head.

Similar to a tongue-cleaner unit 100, a tongue-cleaner unit 200 comprises serration arrays and serration rows. Distinct from a tongue-cleaner unit 100, a tongue-cleaner unit 200 comprises only serration arrays and rows 201 and 220 which are configured to be mild (in nature or level of aggressiveness or mildness) and does not comprise serration arrays and rows which are configured to be aggressive (such as serration arrays and rows 101B and 120B of a tongue-cleaner unit 100).

Referring to FIG. 3H, which provides an example of a mild serration array 201 of a tongue-cleaner unit 200, a mild serration array 201 is, in one embodiment, the same as or similar to a mild serration array 101A exemplified in FIG. 2H, with respect to, e.g., serration troughs, serration depths, serration apexes, array profile edges, and edge slopes. Thus, for the exemplary mild array serration 201, its serration troughs 208, serration depths 212, serration apexes 209, array profile edges 211, and edge slopes 210, are, in terms of configurations, same as or similar to, for the exemplary mild serration array 101A, serration troughs 108, serration depths 112, serration apexes 109, array profile edges 111, and edge slopes 110, respectively.

For an exemplary tongue-cleaner unit 200, a serration row 220 of tongue-cleaner unit 200 comprises a respective bridge 203 which include one or more mild serrations 218 (namely, one or more serrations each configured to be mild), resulting in a serration row 220 realizing a series of continuous mild serrations, which translates to a continuous and soft tongue-cleaning actions (e.g., scraping actions) during an Oral-B-repurposed tongue-cleaning operation.

Referring to FIG. 3I, which is an isometric view of an exemplary bridge 203 of a serration row 220, the exemplary bridge 203 comprises a mild serration 218 which comes in contact with a tongue when a host tongue-cleaner unit 200 (hosting the serration row 220 having the exemplary bridge 203) is properly placed on the tongue while idle during an Oral-B-repurposed tongue-cleaning operation. The exemplary bridge 203 is also configured to have two angled side faces 219 configured to couple the exemplary bridge 203 to respective serration profiles (e.g., two respective serration arrays 201) on the two respective sides of the exemplary bridge 203.

In one embodiment, for the exemplary tongue-cleaner unit 200, the projecting height of a mild serration array 201 of a serration row 220 is configured to be 3 mm or therearound, which is same as, or similar to, that of a serration array 101 of a serration row 120 of an exemplary tongue-cleaner unit 100. Distinct from the projecting height of a bridge 103 of a serration row 120 of an exemplary tongue-cleaner unit 100, the projecting height of a bridge 203 of a serration row 220 is configured to be 3 mm or therearound, which entails that the bridge 203 is in line, in terms of respective projecting heights, with the respective adjacent serration arrays 201 of the serration row 220. This configuration, as relating to the respective projecting heights, results in mild cleaning actions on a tongue, tongue-cleaning actions which prevent or otherwise reduce irritations to the tongue during an Oral-B-repurposed tongue-cleaning operation.

In one embodiment, for the exemplary tongue-cleaner unit 200, similar to an exemplary tongue-cleaner unit 100, one or more debris collection channels 205, which forms instrument row 225, are deployed in between two serration rows 220.

Similar to an exemplary tongue-cleaner unit 100 which comprises symbol 107 signifying one or more natures of the exemplary tongue-cleaner unit 100, an exemplary tongue-cleaner unit 200 also comprises a symbol 207 signifying one or more natures of the exemplary tongue-cleaner unit 200. Distinct from symbol 107 of an exemplary tongue-cleaner unit 100, which, as discussed above, signifies an aggressive nature of an exemplary tongue-cleaner unit 100 with a visual zig-zag pattern, symbol 207 of the exemplary tongue-cleaner unit 200 signifies a mild nature of exemplary tongue-cleaner unit 200 with an exemplary visual pattern formed of three identical circles.

Similar to a tongue-cleaner unit 100, a tongue-cleaner unit 200 may, if space permits on its encompassing base 204 after deployment of its serration rows 220, further comprise one or more instrument rows deployed as the last one or two longitudinally lined instrument rows. In one embodiment, similar to an exemplary tongue-cleaner unit 100, which comprises two last longitudinally lined instrument rows (which, in its case, are two serration rows 121B) horizontally deployed adjacent to a portion of the perimeter of its encompassing base 104, an exemplary tongue-cleaner unit 200 also comprises, in taking advantage of availability of areas remaining in encompassing base 204, two last longitudinally lined instrument rows horizontally deployed adjacent to a portion of the perimeter of its encompassing base 204, except that in its case, the penultimate instrument row is instrument row 222 consisting of debris collection channels 205 and the last instrument row is serration row 221 consisting of one mild serration array 201.

For an exemplary tongue-cleaner unit 200, having the penultimate row 222 serves to provide additional visual feedback of collected debris (that comes with the provision of an additional debris collection channel 205 for the penultimate instrument row 222), a utility of which the relatively mild exemplary tongue-cleaner unit 200 may be in more need of than the relatively aggressive exemplary tongue-cleaner unit 100, and having the last serration row 221 serves to provide an additional tongue-cleaning (e.g., soft-scraping) option as well as additional tongue-cleaning capacity.

In one embodiment, same as or similar to an exemplary tongue-cleaner unit 100, for an exemplary tongue-cleaner unit 200, first, its encompassing base 204 is also configured to be 2 mm or therearound in upright thickness. Second, for each of its serration rows 220, the respective angles between its respective lateral-extending axes of the two respective serration arrays 201 subtended at the respective bridge 203 (which, as exemplified and illustrated, extends horizontally and orthogonal to a longitudinal axis 228 of encompassing base 204), are also uniformly 135 degrees or therearound. Third, the respective longitudinal distance (i.e., transverse distance) between its two longitudinally (transversely) adjacent serration rows 220 (as, e.g., measured between two longitudinally adjacent bridges 203) are uniformly configured to be 5 mm or therearound. Fourth, its encompassing base 204 is configured to be circular or quasi-circular in shape (contour) as well and is configured as well to have the diameter or quasi-diameter of its circular or quasi-circular contour being roughly two times the diameter of the circular brush unit of a commercially available Oral-B toothbrush head.

Hereinafter, a “Sonicare-compatible tongue-cleaner unit” refers to a tongue-cleaner unit of the present disclosure, which is configured and adapted for substituting for the brush unit of an otherwise Sonicare-compatible host brush head and which, with the substitution, repurposes or otherwise transforms, the otherwise Sonicare-compatible host brush head into an advantageous tongue-cleaner head (i.e., a replacement head except for being adapted for tongue-cleaning actions) compatible with a Sonicare electric toothbrush. As a skilled artisan appreciates, a Sonicare-compatible tongue-cleaner unit retains or otherwise comprises, by definition, a set of one or more interface elements of the docking adapter of an otherwise Sonicare-compatible host brush head as discussed above in connection with the related art, the set of one or more interface elements which the substituted brush unit of the otherwise Oral-B-compatible host brush head comprises.

Hereinafter, a “Sonicare-compatible tongue-cleaner head” refers to an otherwise Sonicare-compatible brush head, which, with the substitution of a Sonicare-compatible tongue-cleaner unit for its typically elliptical-shaped brush unit, is repurposed, or otherwise transformed, into an advantageous tongue-cleaner head compatible with a Sonicare electric toothbrush, in accordance with one or more embodiments of the present disclosure.

Hereinafter, a “Sonicare-repurposed tongue-cleaner” refers to an electric tongue-cleaner advantageously formed for performing tongue-cleaning actions, with the advantageous electric tongue-cleaner being an ordered combination of a Sonicare-compatible tongue-cleaner head and the coupling handle of an otherwise host Sonicare electric toothbrush, the ordered combination which is formed through attaching and docking the Sonicare-compatible tongue-cleaner head to the coupling handle through the docking adapter of the Sonicare-compatible tongue-cleaner head such that the Sonicare-compatible tongue-cleaner head, upon being docked, becomes an integral part of the ordered combination in being mobilized by the coupling handle and undergoing, as a result of the mobilization, vibrating and oscillating motions in, as discussed above in connection with the related art, well-known distinct manners (otherwise intended for teeth-cleaning) for performing advantageous tongue-cleaning actions, in accordance with one or more embodiments of the present disclosure.

Hereinafter, “Sonicare-repurposed tongue-cleaning actions” refers to cleaning actions performed on the upper surface or surfaces of the body of a tongue using a Sonicare-repurposed tongue-cleaner, particularly the Sonicare-compatible tongue-cleaner unit thereof (in taking advantage of the vibrating and oscillating motions which the Sonicare-compatible tongue-cleaner unit undergoes in, as discussed above in connection with the related art, well-known distinct manners when being mobilized by the coupling handle of the Sonicare-repurposed tongue-cleaner), with the Sonicare-compatible tongue-cleaner unit being placed against the upper surface or surfaces of the tongue body and properly oriented (i.e., oriented in one or more manners purported for tongue-cleaning), as is exemplified herein in FIG. 2O, in accordance with one or more embodiments of the present disclosure.

Hereinafter, a “Sonicare-repurposed tongue-cleaning operation” refers to a tongue-cleaning operation performed on the upper surface or surfaces of the body of a tongue using an Sonicare-repurposed tongue-cleaner, particularly the Sonicare-compatible tongue-cleaner unit thereof, an operation which, by definition, comprises Sonicare-repurposed tongue-cleaning actions.

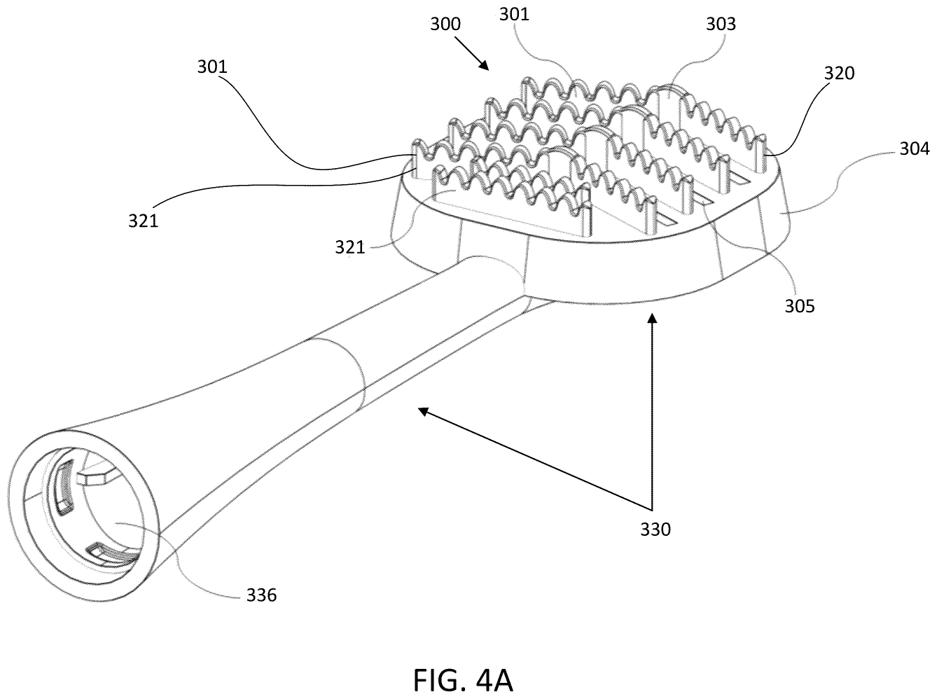

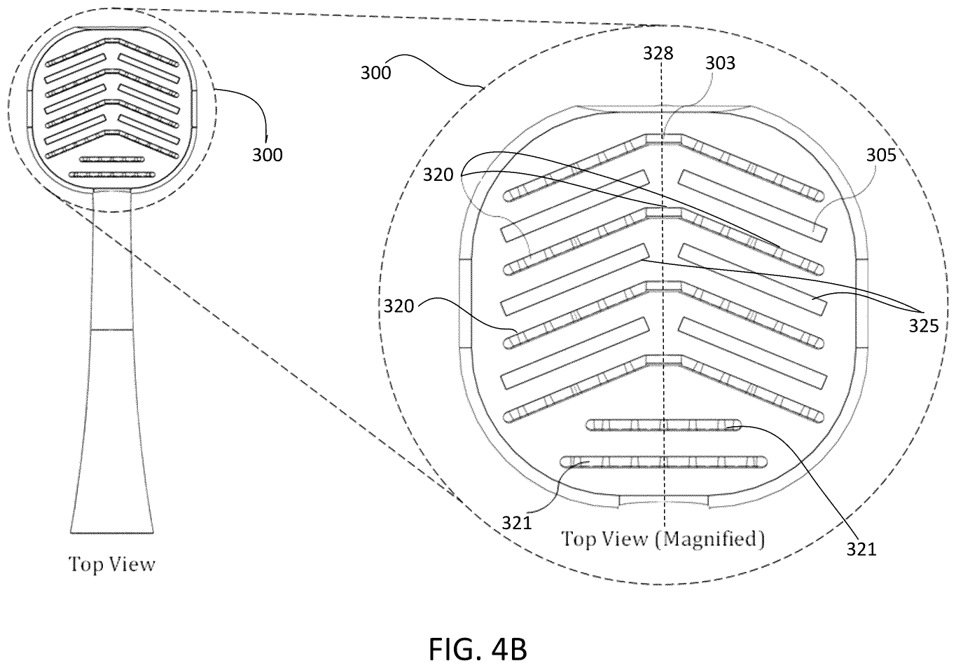

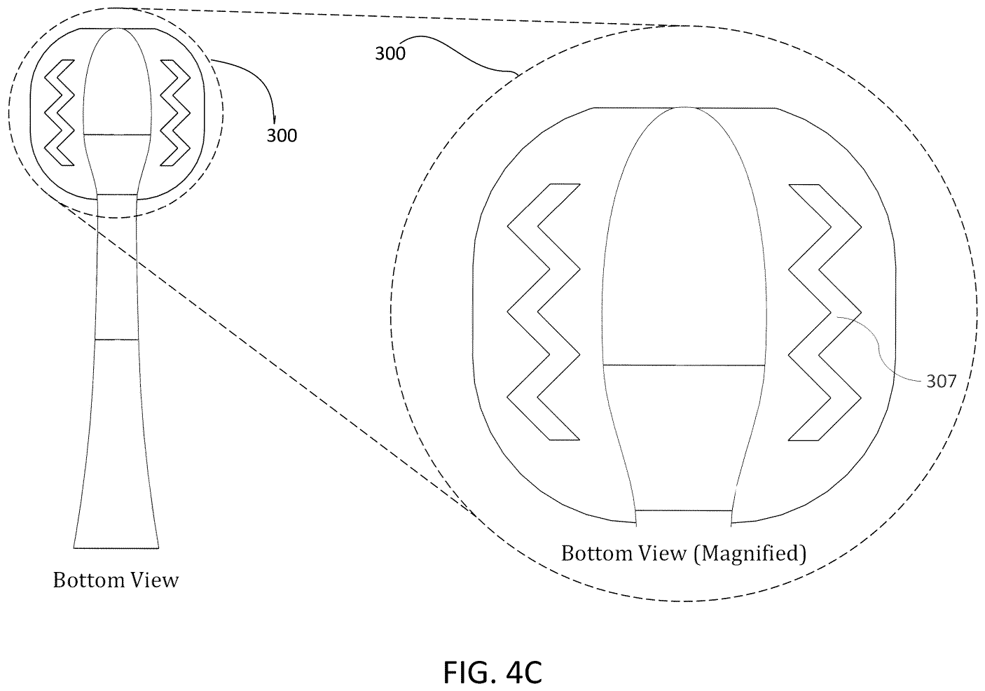

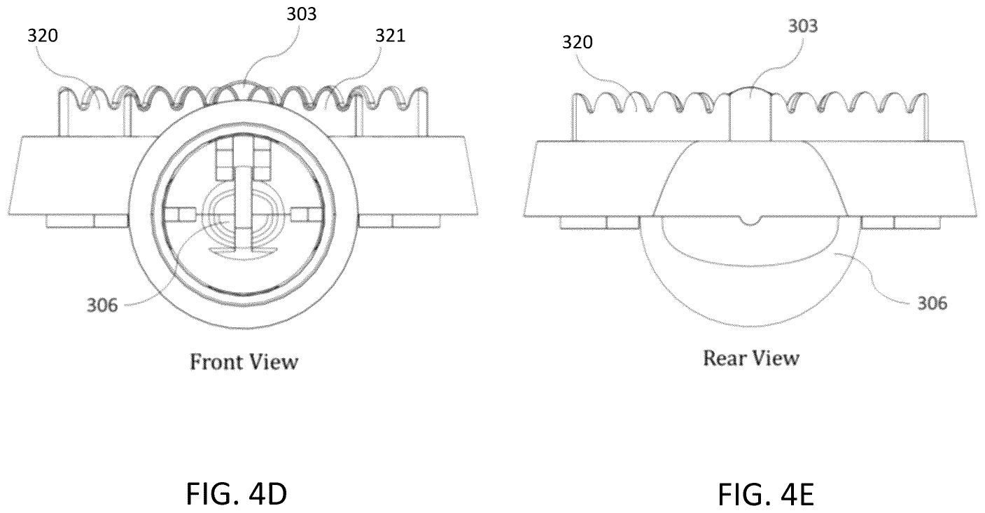

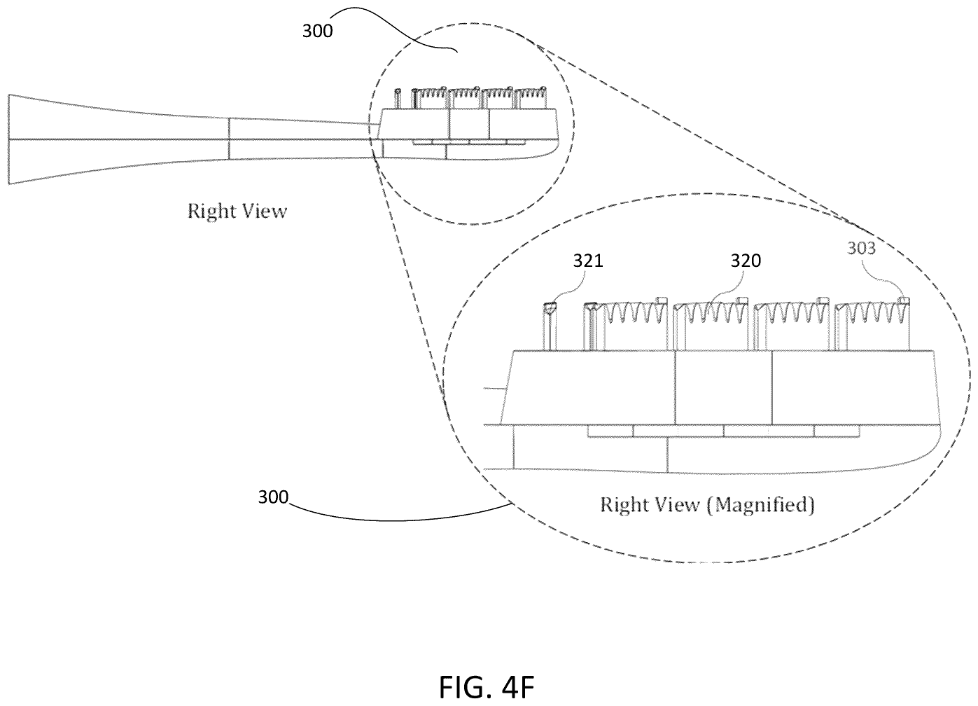

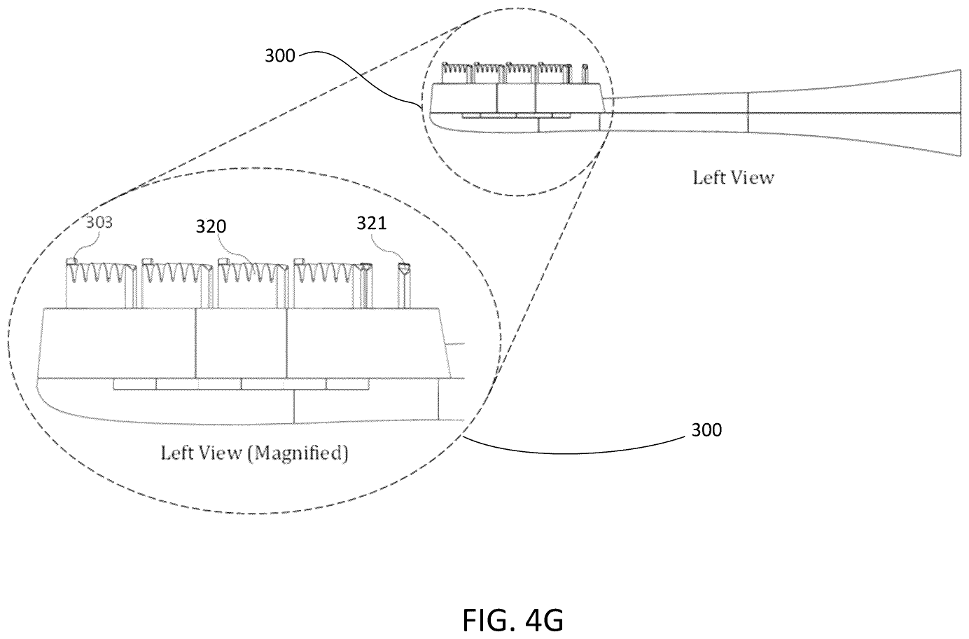

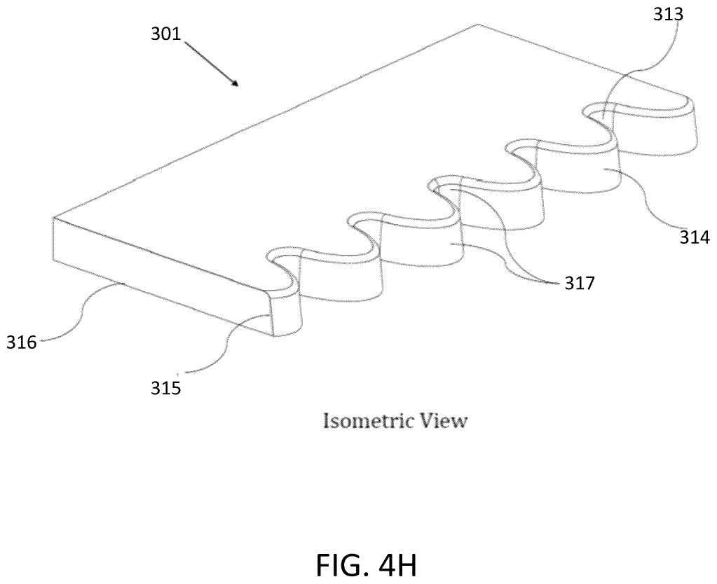

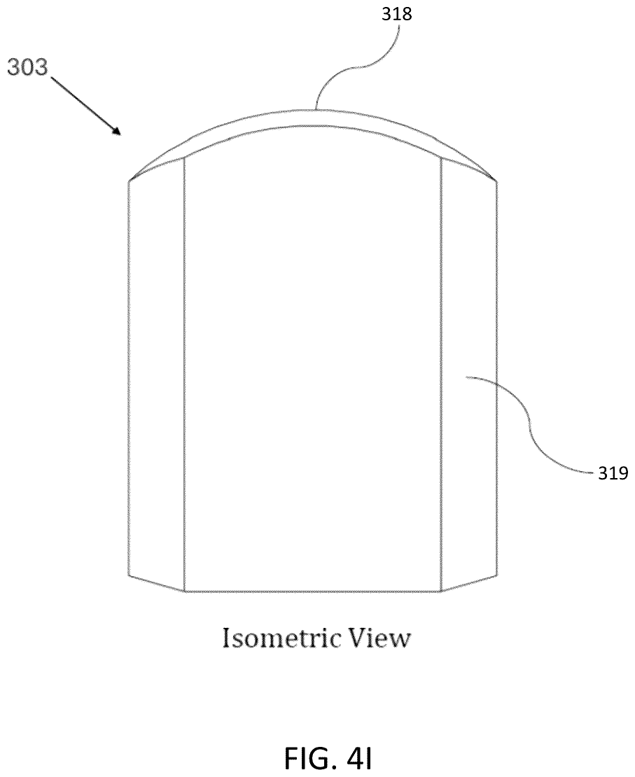

FIGS. 4A-I are different views illustrating an exemplary Sonicare-compatible tongue-cleaner head 330, which includes an exemplary Sonicare-compatible tongue-cleaner unit 300 configured to be an aggressive type of Sonicare-compatible tongue-cleaner unit, according to one or more embodiments of the present disclosure. Hereinafter, a Sonicare-compatible tongue-cleaner unit of an aggressive type will simply be referred to a “tongue-cleaner unit 300.”

Similar to an Oral-B-compatible tongue-cleaner unit 100 (which is configured to have its set of one or more interface elements 106), a Sonicare-compatible tongue-cleaner unit 300 (of a Sonicare-compatible tongue-cleaner head 330) is configured to have a set of well-known one or more interface elements 306, which is an integral part of the well-known docking adapter 336 of a Sonicare-compatible tongue-cleaner head 330. As discussed above, the docking adapter of an otherwise Sonicare-compatible brush head is distinct from the docking adapter of an otherwise Oral-B-compatible brush head. The set of one or more interface elements 306 of a Sonicare-compatible tongue-cleaner unit 300, being distinct from the set of one or more interface elements 106 of an Oral-B-compatible tongue-cleaner unit 100, plays, for its host tongue-cleaner unit 300, the same, or a similar, role which the latter plays for its host tongue-cleaner unit 100.

Similar to an Oral-B-compatible tongue-cleaner unit 100 which comprises an encompassing base 104, a Sonicare-compatible tongue-cleaner unit 300 comprises an encompassing base 304. Although, as discussed above, it is well-known the brush unit of a Sonicare-compatible brush head is distinct from the brush unit of an Oral-B-compatible brush head in terms of, e.g., shape and dimensions, encompassing base 304 may be similar to, or distinct from, encompassing base 104, in terms of shape and dimensions.

Similar to an Oral-B-compatible tongue-cleaner unit 100, a Sonicare-compatible tongue-cleaner unit 300 (hereinafter “tongue-cleaner unit 300”) are also configured to have serration arrays 301 configured to be aggressive.

In one embodiment, distinct from an exemplary aggressive Oral-B-compatible tongue-cleaner unit 100, an exemplary aggressive Sonicare-compatible tongue-cleaner unit 300 is configured to have serration arrays 301, all of which are configured to be aggressive. This “all-aggressive-serration-arrays” configuration takes into account the well-known fact that the brush unit of an otherwise Sonicare-compatible brush head (docked to a host Sonicare toothbrush) undergoes (when the brush unit is being mobilized by a host Sonicare toothbrush), as discussed above in connection with the related art, undergoes linear back-and-forth vibrating and oscillating motions at one or more acoustic or ultrasonic frequencies (as opposed to rotatory, oscillating and pulsating motions which the otherwise brush unit of an otherwise Oral-B-compatible brush head undergoes when being mobilized by a host Oral-B toothbrush).

Referring to FIG. 4H, an exemplary aggressive serration array 301 is configured in a manner same as or similar to an aggressive serration array 101B exemplified in FIG. 2I, with respect to, e.g., its troughs (valleys) 313, apexes 314, serration depths 317, edge slope 315 and array profile edges 316. In one embodiment, the height of an array profile edge 316 is, same as or similar to that of an aggressive serration array 101B exemplified in FIG. 2I, also 5 mm or therearound, of which 3 mm is above encompassing base 304 after the exemplary aggressive serration array 301 is deployed onto encompassing base 304. And the exemplary serration depths 317 are uniformly 1.35 mm or therearound.

Similar to an Oral-B-compatible tongue-cleaner unit 100, a Sonicare-compatible tongue-cleaner unit 300 is also configured to have a respective bridge 303 disposed between two serration arrays 301 for a serration row 320. Referring to FIG. 4I, an exemplary bridge 303 is configured, similar to a bridge 103 exemplified in FIG. 2J, to have a non-serrated apex surface 318 and a pair of respective angled faces 319 through which a respective pair of aggressive serration arrays 301 are joined to the exemplary bridge 303. Referring to FIG. 4B, for each serration row 320, a respective pair of aggressive serration arrays 301 are subtended at a respective bridge 303—which, as exemplified and illustrated, extends horizontally and orthogonal to a longitudinal axis 328 of encompassing base 304—by an obtuse angle, such as 135 degrees.

In one embodiment, same as or similar to bridges 103 of an exemplified tongue-cleaner unit 100, bridges 303 are also each configured to have a projecting height of 3.65 mm or therearound. This configuration likewise entails that the respective non-serrated surface 318 of each exemplary bridges 303 is taller than the pair of respective adjacent serration arrays 301 joined and coupled thereto, resulting in the non-serrated surface 318, rather than apexes of the serrations of the two coupled respective adjacent aggressive serration arrays 301, coming in contact with a tongue (when the exemplary tongue-cleaner unit 300 is properly placed or otherwise deployed on the tongue while idle during a Sonicare-repurposed tongue-cleaning operation), a setting which reduces, if not outright prevents, the possibility of any part of the tongue experiencing any irritation.

In one embodiment, same as or similar to an exemplified tongue-cleaner unit 100, first, an exemplified tongue-cleaner unit 300 is also configured to have a distance of 5 mm or therearound between adjacent serration rows 320. Second, an exemplified tongue-cleaner unit 300 is similarly configured, for each serration row 320, to have an angle of 135 degrees or therearound between its two respective serration arrays 301 subtended at its respective bridge 303. Third, the exemplified tongue-cleaner unit 300 is similarly configured to have one or more instrument rows 325, each consisting of one or more debris-collection channels 305, deployed in between two serration rows 320 deployed longitudinally adjacent to each other. And each exemplary debris-collection channel 305 is configured, similar to each exemplary debris-collection channel 105 of the exemplary tongue-cleaner unit 100, to have a respective recessed receptacle configured to have of a recessed depth of 1.5 mm or therearound, for collecting debris and providing visual feedback to a user during a Sonicare-repurposed tongue-cleaning operation.

Similar to an exemplary tongue-cleaner unit 100 which comprises symbol 107 signifying one or more natures, particularly the aggressive nature there-among, of the exemplary tongue-cleaner unit 100, an exemplary tongue-cleaner unit 300, in one embodiment, also comprises a symbol 307 signifying its one or more natures, particularly its aggressive nature there-among.

Similar to a tongue-cleaner unit 100, a tongue-cleaner unit 300 may, if space permits on its encompassing base 304 after deployment of its serration rows 320, further comprise one or more instrument rows 321 deployed as the last one or two longitudinally lined instrument rows 321. In one embodiment, an exemplary tongue-cleaner unit 300 also comprises two last longitudinally lined serration row 321 each consisting of one aggressive serration array 301.

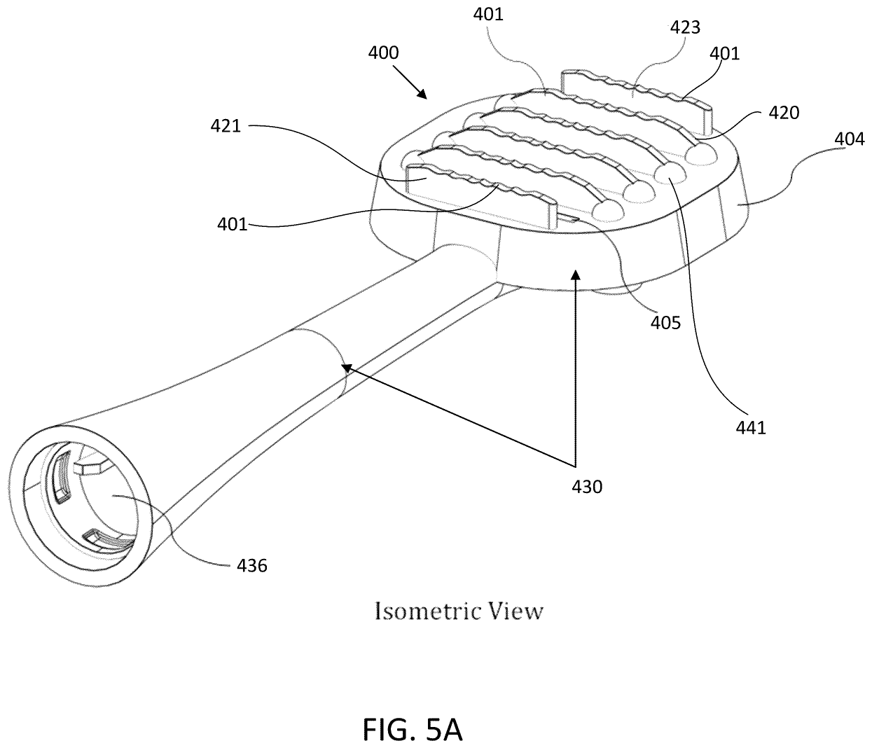

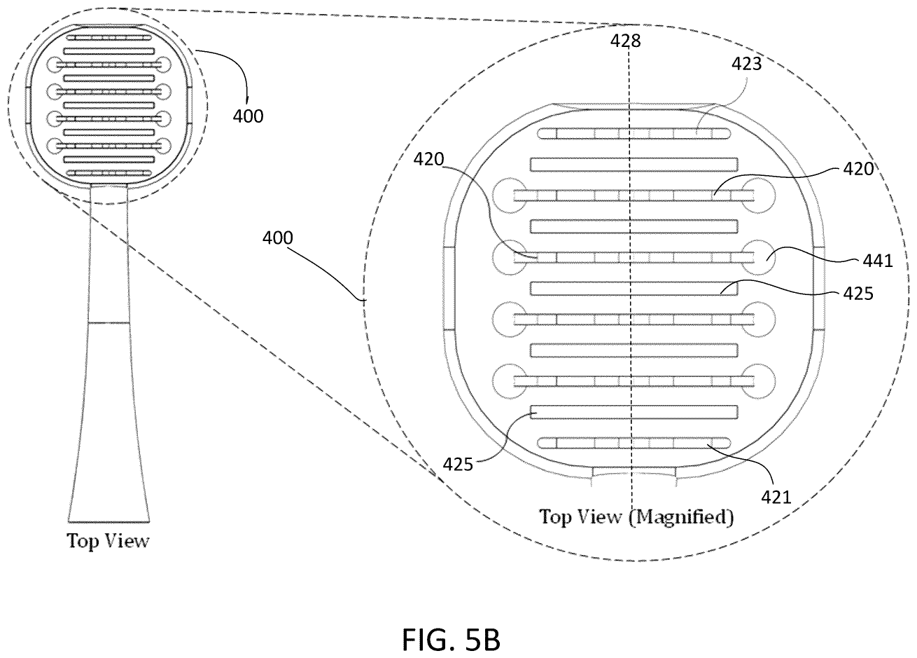

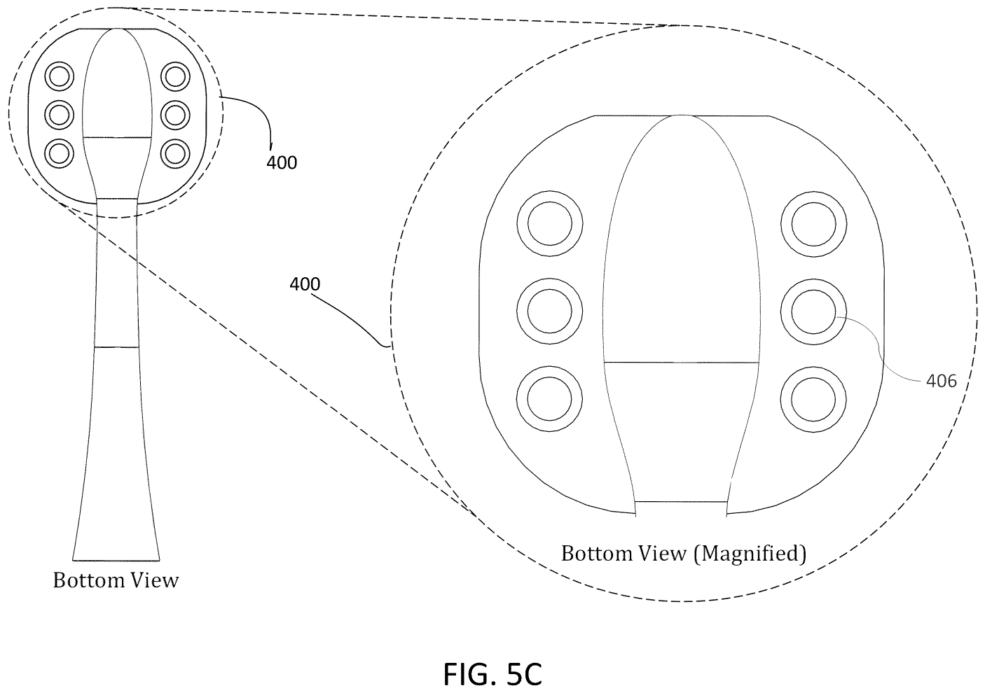

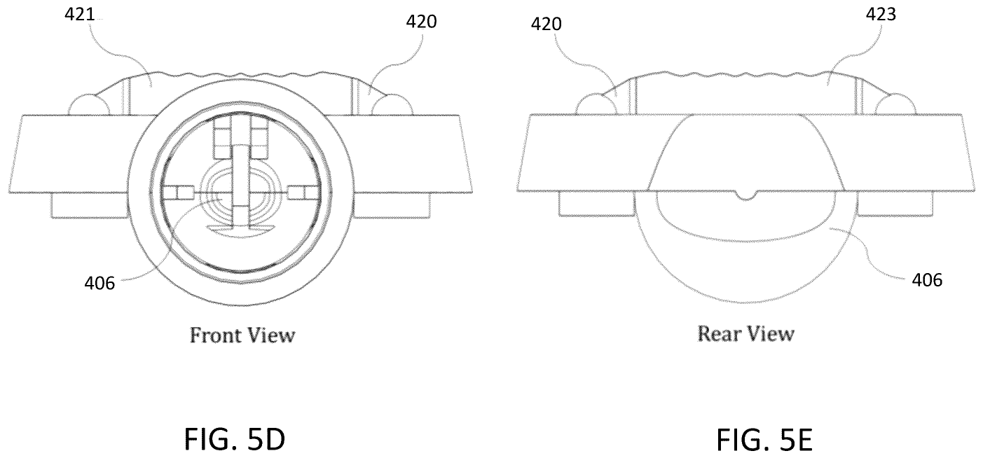

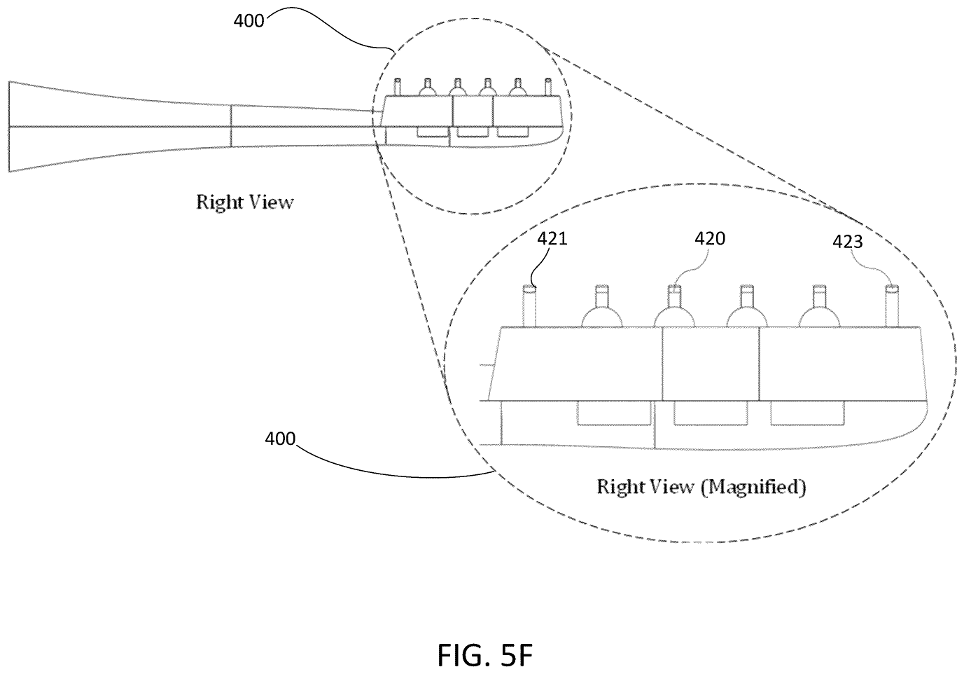

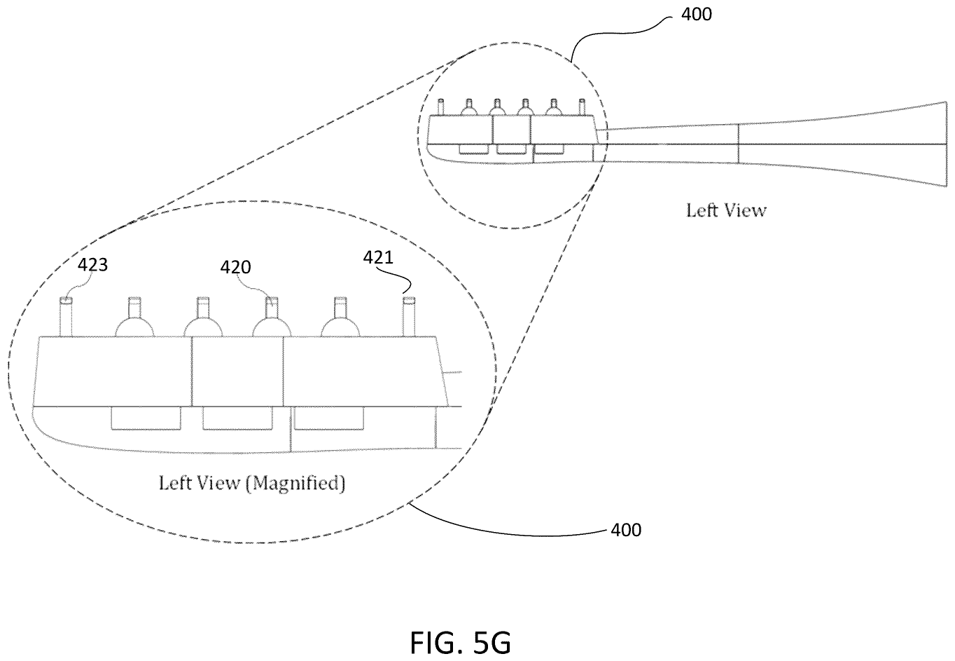

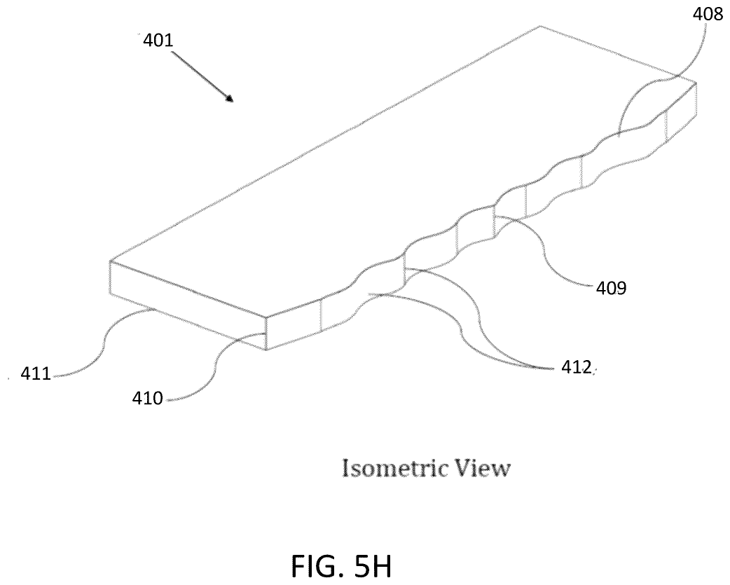

FIGS. 5A-H are perspective views illustrating an exemplary mild type of Sonicare-compatible tongue-cleaner head 430, which includes an exemplary Sonicare-compatible tongue-cleaner unit 400 configured to be a mild type of Sonicare-compatible tongue-cleaner unit, according to one or more embodiments of the present disclosure. Hereinafter, a Sonicare-compatible tongue-cleaner unit of a mild type will simply be referred to a “tongue-cleaner unit 400.”

A mild type of Sonicare-compatible tongue-cleaner head 430, as illustrated, is same as or similar to an aggressive type of Sonicare-compatible tongue-cleaner head 330 in aspects pertaining to their underlying commonalities, which include, inter alia, that they are both a Sonicare-compatible tongue-cleaner head and that they are both adapted to perform a Sonicare-repurposed tongue-cleaning operation. Thus, a Sonicare-compatible tongue-cleaner head 430 is also configured to have the docking adapter 436, which is same as or similar to the docking adapter 336 of a Sonicare-compatible tongue-cleaner head 330.

In the same or a similar token, Sonicare-compatible tongue-cleaner unit 400 (which is configured to be of a mild type), as illustrated, is similar to Sonicare-compatible tongue-cleaner unit 300 (which is configured to be of an aggressive type) in aspects pertaining to their underlying commonalities, which include, inter alia, that they are both a Sonicare-compatible tongue-cleaner unit and that they are both used to perform a Sonicare-repurposed tongue-cleaning operation. Thus, a Sonicare-compatible tongue-cleaner unit 400 is configured to include a set of interface elements 406 (as an integral part of the docking adapter 436 of the tongue-cleaner head 430 hosting the tongue-cleaner unit 400), which is same as or similar to the set of interface elements 306 of a Sonicare-compatible tongue-cleaner unit 300.

Similar to an exemplary Oral-B-compatible tongue-cleaner unit 200, an exemplary Sonicare-compatible tongue-cleaner unit 400 is configured to have serration arrays 401 uniformly configured to be mild in nature or level of aggressiveness or mildness.

Referring to FIG. 5H, an exemplary mild serration array 401 is configured in a manner same as, or similar to, the manner in which an Oral-B-compatible mild serration array 201 exemplified in FIG. 3H is configured, with respect to, e.g., its troughs (valleys) 408, apexes 409, serration depths 412, edge slopes 410 and array profile edges 411. In one embodiment, the height of an exemplary array profile edge 411 is 4.5 mm or therearound, of which 2.5 mm is above encompassing base 404 after the exemplary mild serration array 401 is deployed onto encompassing base 404. And the exemplary serration depths 412 are uniformly 0.3 mm or therearound.

In one embodiment, an exemplary Sonicare-compatible tongue-cleaner unit 400 is configured to have serration rows 420 (each consisting of one serration array 401) uniformly aligned in parallel and orthogonal to a longitudinal axis 428 of its encompassing base 404, a configuration which is distinct from not only an exemplary Sonicare-compatible tongue-cleaner unit 300 configured to be of an aggressive type, but an exemplary mild Oral-B-compatible tongue-cleaner unit 200 configured to be of a mild type. This alignment configuration is provided to take advantage of vibratory and oscillatory motions which a Sonicare-compatible tongue-cleaner unit 400 undergoes during a Sonicare-repurposed tongue-cleaning operation.

In one embodiment, an exemplary Sonicare-compatible tongue-cleaner unit 400 is configured to have scrape bumps 441 deployed to, inter alia, obfuscate array profile edges (including edge corners) of serration arrays 401 of serration rows 420 in advantageously preventing, or otherwise reducing, tongue irritations that a user may otherwise experience during a Sonicare-repurposed tongue-cleaning operation. As another advantage, the provision of scrap bumps 441 enables an exemplary Sonicare-compatible tongue-cleaner unit 400 to perform, as a part of tongue-cleaning actions, flattening or matting actions on tissues of a tongue (during a Sonicare-repurposed tongue-cleaning operation), actions which are instrumental in stimulating tissues of the tongue and giving, e.g., ensuing scraping actions a fresh start.

In one embodiment, no scrap bump 441 is deployed to obfuscate any of the respective edges of the two serration arrays 421 and 423 respectively deployed, space permitting, on both longitudinal ends of encompassing base 404.

Further, an exemplary Sonicare-compatible tongue-cleaner unit 400 is configured to have one or more instrument rows 425 each consisting of one or more debris-collection channels 405 deployed in between two serration rows 420 deployed longitudinally adjacent to each other.

In one embodiment, the longitudinal distance between two serration rows 420 deployed longitudinally adjacent to each other is uniformly 4.5 mm or therearound. And each exemplary debris-collection channel 405 is configured, similar to each exemplary debris-collection channel 305 of the exemplary tongue-cleaner unit 300, to have a respective recessed receptacle configured to have a recessed depth of 1.5 mm or therearound, for collecting debris and providing visual feedback to a user during a Sonicare-repurposed tongue-cleaning operation.

Similar to an exemplary tongue-cleaner unit 200 which comprises symbol 207 signifying one or more natures, particularly the mild nature there-among, of the exemplary tongue-cleaner unit 200, an exemplary tongue-cleaner unit 400, in one embodiment, also comprises a symbol 407 signifying one or more natures, particularly its mild nature there-among.

While the present disclosure has been described with reference to exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure.

A person understanding the present disclosure may now conceive of alternative structures and embodiments or variations of the above, all of which are intended to fall within the scope of the present disclosure as defined in the claims that follow.

What is claimed is:

1. An Oral-B-compatible tongue-cleaner head, the tongue-cleaner head comprises:
an Oral-B-compatible tongue-cleaner unit, the tongue-cleaner unit comprises a set of tongue-cleaning instrument rows configured to perform respective distinct functions pertaining to tongue-cleaning; and
wherein the set of tongue-cleaning instrument rows comprises at least two tongue-cleaning serration rows each configured to be of a respective level of aggressiveness or mildness; and
wherein each of the at least two tongue-cleaning serration rows comprises a tongue-cleaning serration array is configured to have a respective lateral-extending axis and configured, when being used to perform an Oral-B-repurposed tongue-cleaning operation, not to cause the respective lateral-extending axis to get aligned along a vertical axis of a tongue during an Oral-B-repurposed tongue-cleaning operation.
